(19) United States
(12) Patent Application Publication
(10) Pub. No.: US 2026/0366127 A1
(43) Pub. Date: Nov. 19, 2026

(54) DILUTE ZINC ALLOYS

(71) Applicant: Monash University, Clayton (AU)

(72) Inventors: Jian-Feng Nie, Clayton (AU); Chengcheng Wu, Clayton (AU)

(21) Appl. No.: 19/124,389

(22) PCT Filed: Oct. 26, 2023
(86) PCT No.: PCT/AU2023/051079

§ 371 Date: Apr. 25, 2025

(30) Foreign Application Priority Data
Oct. 28, 2022 (AU) 2022903205

(51) Int. Cl. C22F 1/16; A61L 31/02; C22C 18/00; C22C 18/02

(52) U.S. Cl. CPC C22F 1/165; A61L 31/022; C22C 18/00; C22C 18/02

(57) ABSTRACT

Dilute zinc alloys and processes for their manufacture are provided. The alloys contain iron and/or copper as major alloying metals and are prepared using hot extrusion and optional post-extrusion annealing. The alloys exhibit high strength and improved creep resistance. The alloys find particular, although not exclusive, use in the fabrication of biodegradable medical implants.

### FIELD OF THE DISCLOSURE

The present disclosure relates to dilute zinc alloys and processes for their manufacture. The alloys exhibit high strength and improved creep resistance, and find particular, although not exclusive, use in the fabrication of biodegradable medical implants.

### BACKGROUND OF THE DISCLOSURE

Over the past several decades, zinc alloys having non-toxic compositions have received increasing attention for fabricating biodegradable products because of their intrinsic advantages in load-bearing bio-applications. First, as an emerging class of metallic biomaterials, zinc alloys generally have better mechanical properties for bio-scaffolding applications than their polymeric counterparts. Second, compared with magnesium and iron based metallic biomaterials, zinc alloys have an intermediate biodegradation rate, which better matches the healing rate of, for example, human bones. Furthermore, as zinc is known to be an essential element in humans, participating in nucleic acid metabolism and other basic biological processes, zinc alloys are considered to have good biocompatibility and biosafety. However, inadequate strength and creep resistance of biocompatible zinc alloys at human body temperature remain a major issue to their use in biodegradable applications.

Alloying and thermomechanical processing are the two typical approaches to improve mechanical properties of zinc alloys. However, as most biocompatible elements have low solubilities in a zinc matrix, the addition of a small amounts of biocompatible elements does not effectively improve the strength of zinc alloys, and the addition of an excess amount of alloying elements causes formation of intermetallic particles that deteriorate both strength and ductility of zinc alloys. The application of thermomechanical processing to zinc alloys improves strength by grain refinement. However, fine-grained microstructure in zinc alloys causes acceleration of bio-corrosion rate and creep deformation rate at human body temperature, which is harmful in biodegradable material applications.

Therefore, it would be desirable to provide zinc alloys containing dilute amounts of alloying elements and processes for their manufacture such that high strength is achieved without accelerating creep rate.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE DISCLOSURE

In one aspect the present disclosure provides a process for preparing a dilute zinc alloy comprising the steps of:
extruding homogenised dilute zinc alloy at a temperature above 175 °C; and
optionally annealing the extruded dilute zinc alloy at a temperature from about 250 °C to about 400 °C;
wherein the dilute zinc alloy comprises at least 99.0 wt. % zinc; and one or both of iron and copper in total amount from about 0.01 wt. % to about 0.99 wt. %;
wherein the dilute zinc alloy comprises from about 0.01 wt. % to about 0.7 wt. % of one or more of magnesium, calcium, manganese, and lithium; and
wherein the sum of iron, copper, and one or more of magnesium, calcium, manganese, and lithium does not exceed about 1.0 wt. %.

In embodiments the dilute zinc alloy comprises at least 99.0 wt. % zinc; and one or both of iron and copper in total amount from about 0.03 wt. % to about 0.78 wt. %;
wherein the dilute zinc alloy comprises from about 0.02 wt. % to about 0.7 wt. % of one or more of magnesium, calcium, manganese, and lithium; and
wherein the sum of iron, copper, and one or more of magnesium, calcium, manganese, and lithium does not exceed 0.8 wt. %.

In embodiments, the dilute zinc alloy comprises at least 99.0 wt. % zinc; and either,
from about 0.02 wt. % to about 0.97 wt. % iron and from about 0.01 wt. % to about 0.1 wt. % copper, or
from about 0.06 wt. % to about 0.5 wt. % copper and from about 0.01 wt. % to about 0.19 wt. % iron;
wherein the dilute zinc alloy comprises from about 0.01 wt. % to about 0.2 wt. % of one or more of magnesium, calcium, manganese, and lithium; and
wherein the sum of iron, copper, and one or more of magnesium, calcium, manganese, and lithium does not exceed 1.0 wt. %.

In embodiments, the dilute zinc alloy comprises at least 99.5 wt. % zinc; and either,
from about 0.02 wt. % to about 0.5 wt. % iron and from about 0.01 wt. % to about 0.06 wt. % copper, or
from about 0.06 wt. % to about 0.2 wt. % copper and from about 0.01 wt. % to about 0.05 wt. % iron;
wherein the dilute zinc alloy comprises from about 0.01 wt. % to about 0.2 wt. % of one or more of magnesium, calcium, manganese, and lithium; and
wherein the sum of iron, copper, and one or more of magnesium, calcium, manganese, and lithium does not exceed 0.5 wt. %.

In embodiments of the process, the sum of iron, copper, and one or more of magnesium, calcium, manganese, and lithium does not exceed 0.7 wt. %, or 0.6 wt. %, or 0.5 wt. %, or 0.4 wt. %, or 0.3 wt. %.

In embodiments, the dilute zinc alloy has a strong basal texture post-extrusion.

In embodiments, the dilute zinc alloy has a strong basal-texture post annealing.

In embodiments, extrusion is performed at a temperature above 200 °C.

In embodiments, extrusion is performed at a temperature from about 200 °C to about 380 °C.

In embodiments, extrusion speed is from about 0.01 mm·s⁻¹ to about 10 mm·s⁻¹, or from about 0.05 mm·s⁻¹ to about 0.5 mm·s⁻¹

In embodiments, extrusion ratio is from about 8:1 to about 80:1, or from about 19:1 to about 50:1.

In embodiments, the annealing is performed at a temperature from about 250 °C to about 350 °C.

In embodiments, the annealing is performed from about 0.5 hours to about 50 hours, or from about 1 hour to about 10 hours.

In embodiments, the process comprises a homogenisation step prior to extrusion wherein the dilute zinc alloy is maintained at a temperature from about 300 °C to about 400 °C. The homogenisation may be conducted for a period up to about 5 hours.

In embodiments, an average grain size of the post-extruded dilute zinc alloy is greater than about 5 micron, or greater than about 10 micron.

In embodiments, an average grain size of the post-extruded dilute zinc alloy is less than about 25 micron.

In embodiments, the annealing process increases an average grain size of the dilute zinc alloy.

In embodiments, an average grain size of the post-annealed dilute zinc alloy is greater than about 25 micron.

In embodiments, an average grain size of the post-annealed dilute zinc alloy is from about 25 micron to about 1000 micron.

In embodiments, the annealing process increases the dilute zinc alloy's ambient temperature compressive yield strength.

In embodiments, the annealing process decreases the dilute zinc alloy's minimum creep rate.

In embodiments of the process, the dilute zinc alloy comprises:
(a) at least 99.0 wt. % zinc;
(b) about 0.01 wt. % iron;
(c) about 0.04 wt. % copper; and
(d) about 0.05 wt. % magnesium;
   or
(a) at least 99.0 wt. % zinc;
(b) about 0.06 wt. % copper; and
(c) about 0.04 wt. % magnesium;
   or
(a) at least 99.0 wt. % zinc;
(b) about 0.06 wt. % iron;
(c) about 0.14 wt. % copper;
(d) about 0.01 wt. % calcium;
(e) about 0.09 wt. % manganese; and
(f) about 0.16 wt. % magnesium;
   or
(a) at least 99.0 wt. % zinc;
(b) about 0.01 wt. % iron;
(c) about 0.37 wt. % copper;
(d) about 0.05 wt. % manganese; and
(e) about 0.18 wt. % magnesium;
   or
(a) at least 99.0 wt. % zinc;
(b) about 0.02 wt. % iron;
(c) about 0.1 wt. % copper;
(d) about 0.02 wt. % calcium;
(e) about 0.16 wt. % manganese; and
(f) about 0.15 wt. % magnesium
   or
(a) about 99.6 wt. % zinc;
(b) about 0.14 wt. % iron;
(c) about 0.05 wt. % copper;
(d) about 0.05 wt. % calcium; and
(e) about 0.05 wt. % magnesium;
   or
(a) about 99.6 wt. % zinc;
(b) about 0.14 wt. % copper;
(c) about 0.03 wt. % iron;
(d) about 0.05 wt. % calcium; and
(e) about 0.05 wt. % magnesium.

In embodiments, the dilute zinc alloy comprises at least 99.1 wt. % zinc, or at least 99.2 wt. % zinc, or at least 99.3 wt. % zinc, or at least 99.4 wt. % zinc, or at least 99.5 wt. % zinc, or at least 99.6 wt. % zinc, or at least 99.7 wt. % zinc.

In another aspect the present disclosure provides a dilute zinc alloy formed by the process according to any one of the herein disclosed embodiments.

In another aspect the present disclosure provides a dilute zinc alloy comprising:
at least 99.0 wt. % zinc; and one or both of iron and copper in total amount from about 0.01 wt. % to about 0.99 wt. %;
wherein the dilute zinc alloy comprises from about 0.01 wt. % to about 0.7 wt. % of one or more of magnesium, calcium, manganese, and lithium; and
wherein the sum of iron, copper, and one or more of magnesium, calcium, manganese, and lithium does not exceed about 1.0 wt. %.

In embodiments, the dilute zinc alloy comprises:
at least 99.0 wt. % zinc; and one or both of iron and copper in total amount from about 0.03 wt. % to about 0.78 wt. %;
wherein the dilute zinc alloy comprises from about 0.02 wt. % to about 0.7 wt. % of one or more of magnesium, calcium, manganese, and lithium; and
wherein the sum of iron, copper, and one or more of magnesium, calcium, manganese, and lithium does not exceed 0.8 wt. %.

In embodiments, the dilute zinc alloy comprises:
(a) at least 99.0 wt. % zinc;
(b) either, from about 0.02 wt. % to about 0.97 wt. % iron and from about 0.01 wt. % to about 0.1 wt. % copper, or from about 0.06 wt. % to about 0.5 wt. % copper and from about 0.01 wt. % to about 0.19 wt. % iron;
(c) from about 0.01 wt. % to about 0.12 wt. % of one or more of magnesium, calcium, manganese, and lithium;
wherein the sum of iron, copper, and one or more of magnesium, calcium, manganese, and lithium does not exceed 1.0 wt. %.

In embodiments, the dilute zinc alloy comprises:
(a) at least 99.5 wt. % zinc;
(b) either, from about 0.02 wt. % to about 0.5 wt. % iron and from about 0.01 wt. % to about 0.06 wt. % copper, or from about 0.06 wt. % to about 0.2 wt. % copper and from about 0.01 wt. % to about 0.05 wt. % iron;
(c) from about 0.01 wt. % to about 0.12 wt. % of one or more of magnesium, calcium, manganese, and lithium;
wherein the sum of iron, copper, and one or more of magnesium, calcium, manganese, and lithium does not exceed 0.5 wt. %.

In embodiments, the sum of iron, copper, and one or more of magnesium, calcium, manganese, and lithium does not exceed 0.7 wt. %, or 0.6 wt. %, or 0.5 wt. %, or 0.4 wt. %, or 0.3 wt. %.

In embodiments the dilute zinc alloy has a strong basal texture.

In embodiments, the average grain size of the dilute zinc alloy is greater than 5 micron, or greater than 10 micron, or greater than 25 micron.

In embodiments, the average grain size of the dilute zinc alloy is from about 5 micron to about 1000 micron.

In embodiments the compressive yield strength of the dilute zinc alloy is greater than 220 MPa when measured at ambient temperature and a strain rate of 10⁻³ s⁻¹.

In embodiments, the minimum compressive creep rate of the dilute zinc alloy is less than 3×10⁻⁶ s⁻¹ under a loading stress of 200 M Pa at 37 °C.

In embodiments, the dilute zinc alloy comprises:
(a) at least 99.0 wt. % zinc;
(b) about 0.01 wt. % iron;
(c) about 0.04 wt. % copper; and
(d) about 0.05 wt. % magnesium;
   or
(a) at least 99.0 wt. % zinc;
(b) about 0.06 wt. % copper; and
(c) about 0.04 wt. % magnesium;
   or
(a) at least 99.0 wt. % zinc;
(b) about 0.06 wt. % iron;
(c) about 0.14 wt. % copper;
(d) about 0.01 wt. % calcium;
(e) about 0.09 wt. % manganese; and
(f) about 0.16 wt. % magnesium;
   or
(a) at least 99.0 wt. % zinc;
(b) about 0.01 wt. % iron;
(c) about 0.37 wt. % copper;
(d) about 0.05 wt. % manganese; and
(e) about 0.18 wt. % magnesium;
   or
(a) at least 99.0 wt. % zinc;
(b) about 0.02 wt. % iron;
(c) about 0.1 wt. % copper;
(d) about 0.02 wt. % calcium;
(e) about 0.16 wt. % manganese; and
(f) about 0.15 wt. % magnesium;
   or
(a) about 99.6 wt. % zinc;
(b) about 0.14 wt. % iron;
(c) about 0.05 wt. % copper;
(d) about 0.05 wt. % calcium; and
(e) about 0.05 wt. % magnesium;
   or
(a) about 99.6 wt. % zinc;
(b) about 0.14 wt. % copper;
(c) about 0.03 wt. % iron;
(d) about 0.05 wt. % calcium; and
(e) about 0.05 wt. % magnesium.

In embodiments, the dilute zinc alloy comprises at least 99.1 wt. % zinc, or at least 99.2 wt. % zinc, or at least 99.3 wt. % zinc, or at least 99.4 wt. % zinc, or at least 99.5 wt. % zinc, or at least 99.6 wt. % zinc, or at least 99.7 wt. % zinc.

In another aspect the present disclosure provides an article of manufacture comprising the dilute zinc alloy according to any one of the herein disclosed embodiments.

In embodiments, the article is a medical implant.

In embodiments, the medical implant is a stent, a plate, a screw, a pin, a nail, or a scaffold.

In embodiments, the medical implant may be a load-bearing structural implant.

In embodiments, the medical implant may be a non-load-bearing structural implant.

Advantageously, the presently disclosed dilute zinc alloys possess high compressive yield strength and improved creep resistance at normal human body temperature.

Advantageously, the presently disclosed dilute zinc alloys degrade at normal human body temperature to release the constituent metals at daily doses which are orders of magnitude lower than the recommended daily intake values for these metals.

Further, in vitro cytotoxicity testing following ISO 10993-5 (2009 Jun. 1) and ISO 10993-12 (2012 Jul. 1) indicated non-toxic responses to cells exposed to extracts of the herein disclosed dilute zinc alloys.

Any embodiment herein shall be taken to apply mutatis mutandis to any other embodiment unless specifically stated otherwise.

The present disclosure is not to be limited in scope by the specific embodiments described herein, which are intended for the purpose of exemplification only. Functionally-equivalent products, compositions and processes are clearly within the scope of the disclosure, as described herein.

Further aspects of the present disclosure and further embodiments of the aspects described in the preceding paragraphs will become apparent from the following description, given by way of example and with reference to the accompanying drawings.

### BRIEF DESCRIPTION OF THE DRAWINGS

FIG. 1 shows electron backscatter diffraction (EBSD) images (left hand images) and grain size distributions (right hand images) of dilute zinc alloys prepared by hot-extrusion at 250 °C and subsequent annealing treatments, according to embodiments of the present disclosure; images (a) no annealing treatment, EBSD scale 100 μm; images (b) 275 °C annealing treatment, EBSD scale 100 μm; images (c) 300 °C annealing treatment, EBSD scale 100 μm; images (d) 350 °C annealing treatment, EBSD scale 200 μm.

FIG. 2 shows textures of dilute zinc alloys prepared by hot-extrusion at 250 °C and subsequent annealing treatments, according to embodiments of the present disclosure.

FIG. 3 shows the room temperature true compressive stress-strain curves of dilute zinc alloys prepared by hot-extrusion at 250 °C and subsequent annealing treatments, according to embodiments of the present disclosure.

FIG. 4 shows compressive creep curves of dilute zinc alloys prepared by hot-extrusion at 250 °C and a subsequent annealing treatment at 350 °C for 2 hours under 200 MPa at 37 °C (human body temperature), according to embodiments of the present disclosure.

FIG. 5 shows E BSD images (left hand images) and grain size distributions (right hand images) of dilute zinc alloys prepared by hot-extrusion at 250 °C and subsequent annealing treatments, according to embodiments of the present disclosure; images (a) no annealing treatment, EBSD scale 100 μm; images (b) 275 °C annealing treatment, EBSD scale 500 μm; images (c) 300 °C annealing treatment, EBSD scale 500 μm; images (d) 350 °C annealing treatment, EBSD scale 1000 μm.

FIG. 6 shows textures of dilute zinc alloys prepared by hot-extrusion at 250 °C and subsequent annealing treatments, according to embodiments of the present disclosure.

FIG. 7 shows the room temperature true compressive stress-strain curves of dilute zinc alloys prepared by hot-extrusion at 250 °C and subsequent annealing treatments, according to embodiments of the present disclosure.

FIG. 8 shows compressive creep curves of dilute zinc alloys prepared by hot-extrusion at 250 °C and a subsequent annealing treatment of 350 °C for 2 hours under 200 MPa at 37 °C (human body temperature), according to embodiments of the present disclosure.

FIG. 9 shows EBSD images (left hand images) and grain size distributions (right hand images) of dilute zinc alloys prepared by hot-extrusion at two extrusion temperatures of 250 °C and 300 °C and subsequent annealing treatments, according to embodiments of the present disclosure; images (a) Extrusion at 250 °C without annealing treatment, EBSD scale 100 μm; images (b) 350 °C annealing treatment after 250 °C extrusion, EBSD scale 100 μm; images (c) extrusion at 300 °C without annealing treatment, EBSD scale 100 μm; images (d) 350 °C annealing treatment after 300 °C extrusion, EBSD scale 500 μm.

FIG. 10 shows textures of dilute zinc alloys prepared by hot-extrusion at two extrusion temperatures of 250 °C and 300 °C and subsequent annealing treatments, according to embodiments of the present disclosure.

FIG. 11 shows the room temperature true compressive stress-strain curves of dilute zinc alloys prepared by hot-extrusion at two extrusion temperatures of 250 °C and 300 °C and subsequent annealing treatments, according to embodiments of the present disclosure.

FIG. 12 shows compressive creep curves of dilute zinc alloys prepared by hot-extrusion at 250 °C and a subsequent annealing treatment of 350 °C for 2 hours, under 250 MPa at 37 °C (human body temperature), according to embodiments of the present disclosure.

FIG. 13 shows EBSD images (left hand images) and grain size distributions (right hand images) of dilute zinc alloys prepared by hot-extrusion at two extrusion temperatures of 250 °C and 300 °C and subsequent annealing treatments, according to embodiments of the present disclosure; images (a) Extrusion at 250 °C without annealing treatment, EBSD scale 50 μm; images (b) 350 °C annealing treatment after 250 °C extrusion, EBSD scale 200 μm; images (c) extrusion at 300 °C without annealing treatment, EBSD scale 100 μm; images (d) 350 °C annealing treatment after 300 °C extrusion, EBSD scale 500 μm.

FIG. 14 shows textures of dilute zinc alloys prepared by hot-extrusion at two extrusion temperatures of 250 °C and 300 °C and subsequent annealing treatments, according to embodiments of the present disclosure.

FIG. 15 shows the room temperature true compressive stress-strain curves of dilute zinc alloys prepared by hot-extrusion at two extrusion temperatures of 250 °C and 300 °C and subsequent annealing treatments, according to embodiments of the present disclosure.

FIG. 16 shows compressive creep curves of dilute zinc alloys prepared by hot-extrusion at 250 °C without annealing treatment and alloys prepared by hot-extrusion at 300 °C and a subsequent annealing treatment of 350 °C for 2 hours, under 250 MPa at 37 °C (human body temperature), according to embodiments of the present disclosure.

FIG. 17 shows EBSD images (left hand images) and grain size distributions (right hand images) of dilute zinc alloys prepared by hot-extrusion at 300 °C and subsequent annealing treatments, according to embodiments of the present disclosure; images (a) no annealing treatment, EBSD scale 100 μm; images (b) 350 °C annealing treatment for 0.5 hour, E BSD scale 100 μm; images (c) 350 °C annealing treatment for 2 hours, E BSD scale 100 μm; images (d) 370 °C annealing treatment for 3 hours, E BSD scale 500 μm.

FIG. 18 shows textures of dilute zinc alloys prepared by hot-extrusion at 300 °C and subsequent annealing treatments, according to embodiments of the present disclosure.

FIG. 19 shows the room temperature true compressive stress-strain curves of dilute zinc alloys prepared by hot-extrusion at 300 °C and subsequent annealing treatments, according to embodiments of the present disclosure.

FIG. 20 shows compressive creep curves of dilute zinc alloys prepared by hot-extrusion at 300 °C and a subsequent annealing treatment of 370 °C for 3 hours, under 250 MPa at 37 °C (human body temperature), according to embodiments of the present disclosure.

FIG. 21 shows the estimated daily doses of six elements of Zn, Mg, Cu, Fe, Mn, and Ca released from the dilute zinc alloys of M5, M6, and M7 in in-vitro biodegradation tests, assuming an implant surface area of 400 mm². These estimated values (captured within lower oval) are compared against recommended daily intake values of these six elements for different human groups (captured within upper oval).

FIG. 22 shows the cell viability of MG-63 osteoblast cells exposed to extracts of M5, M6, and M7 dilute zinc alloys and Mg-0.4Zn-0.1Ca alloy, as a comparative material, at 8 different concentrations for 24 hours. The horizontal dashed line indicates the threshold between toxic and non-toxic responses (70% cell viability), as per ISO 10993-5 (2009 Jun. 1).

FIG. 23 shows the room temperature true compressive stress-strain curves of dilute zinc alloys prepared by hot-extrusion at 250 °C and 350 °C.

FIG. 24 shows the room temperature true compressive stress-strain curves of dilute zinc alloys prepared by hot-extrusion at 250 °C and 350 °C.

FIG. 25 shows textures of dilute zinc alloys (a) extruded at 175 °C, and (b) prepared by hot-extrusion at 175 °C and a subsequent annealing treatment at 300 °C for 2 hours.

FIG. 26 shows textures of dilute zinc alloys (a) extruded at 165 °C, and (b) prepared by hot-extrusion at 165 °C and a subsequent annealing treatment at 300 °C for 2 hours.

FIG. 27 shows the room temperature true compressive stress-strain curves of dilute zinc alloys prepared by hot-extrusion at 175 °C and subsequent annealing treatments.

FIG. 28 shows the room temperature true compressive stress-strain curves of dilute zinc alloys prepared by hot-extrusion at 165 °C and subsequent annealing treatments.

FIG. 29 shows compressive creep curves of a comparative Mg-0.4Zn-0.1Ca alloy prepared by hot-extrusion at 220 °C or 400 °C, under 250 MPa at 37 °C.

FIG. 30 shows EBSD images (left hand images) and grain size distributions (right hand images) of pure zinc prepared by hot-extrusion at 150 °C and subsequent annealing treatment; images (a) no annealing treatment, EBSD scale 200 μm; images (b) 200 °C annealing treatment for 0.5 hour, EBSD scale 200 μm.

FIG. 31 shows textures of pure zinc prepared by hot-extrusion at 150 °C and subsequent annealing treatment.

FIG. 32 shows the room temperature true compressive stress-strain curves of pure zinc prepared by hot-extrusion at 150 °C (compressive yield strength=74 MPa) and subsequent annealing treatment (compressive yield strength=132 MPa).

### DETAILED DESCRIPTION OF THE EMBODIMENTS

It will be understood that the disclosure described and defined in this specification extends to all alternative combinations of two or more of the individual features mentioned or evident from the text or drawings. All of these different combinations constitute various alternative aspects of the disclosure.

### DEFINITIONS

For purposes of interpreting this specification, terms used in the singular will also include the plural and vice versa.

As used herein, except where the context requires otherwise, the term “comprise” and variations of the term, such as “comprising”, “comprises” and “comprised”, are not intended to exclude further additives, components, integers or steps.

“About” as used herein when referring to a measurable value such as an amount, a temporal duration, and the like, is meant to encompass variations of ±20% or ±10%, in some instances ±5%, in some instances ±1%, and in some instances ±0.1% from the specified value, as such variations are appropriate to perform the disclosed processes.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein the term “strong basal texture” in reference to a metal alloy means that the majority of the grains in the metal alloy have their c-axes lying perpendicular to the extrusion direction.

The present disclosure relates to new dilute zinc alloys and processes for their manufacture.

The present disclosure provides biodegradable zinc alloys that have dilute additions of selected alloying elements and processes for manufacturing them. The dilute zinc alloys have high compressive yield strength and improved creep resistance. The processes involve extrusion under specific processing conditions and, optionally, post-extrusion annealing treatments to obtained specific microstructures. Stronger and more creep resistant zinc alloys according to the present disclosure comprise iron and/or copper, with the total amount of added alloying elements less than 1.0 wt. %, and the balance being zinc and inevitable impurity elements (the total content of impurity elements is typically less than 0.06 wt. % and individual impurity element typically less than 0.016 wt. %). These alloys are subjected to hot extrusion at temperatures above 175 °C with appropriate extrusion ratios and speeds to obtain a strong basal texture and optional subsequent annealing at temperature from 250-400 °C. The compressive yield strength of the alloys increases with an increase in grain size caused by post-extrusion annealing at a high temperature or by an appropriate combination of extrusion parameters, which is in contrast to the conventional annealing softening behaviour of a metal or the traditional view that a metal having an enlarged grain size will have a reduced strength.

### Manufacturing Process

In an aspect there is provided a process for preparing a dilute zinc alloy comprising the steps of:
extruding homogenised dilute zinc alloy at a temperature above 175 °C; and
optionally annealing the extruded dilute zinc alloy at a temperature from about 250 °C to about 400 °C;
wherein the dilute zinc alloy comprises at least 99.0 wt. % zinc; and one or both of iron and copper in total amount from about 0.01 wt. % to about 0.99 wt. %;
wherein the dilute zinc alloy comprises from about 0.01 wt. % to about 0.7 wt. % of one or more of magnesium, calcium, manganese, and lithium; and
wherein the sum of iron, copper, and one or more of magnesium, calcium, manganese, and lithium does not exceed about 1.0 wt. %.

Without being bound by theory, an important effect of hot extrusion is to obtain a fully recrystallised microstructure that has a strong basal texture to allow intergranular deformation to be the predominant deformation mode during plastic deformation. The control of this intergranular deformation mode is likely important for annealing strengthening to occur in such dilute alloys or even pure zinc. This is in contrast to the traditional approaches that involve the control of intragranular deformation modes (dislocation slip or twinning), in which the compressive strength decreases with an increase in grain size, following the conventional Hall-Petch relationship and the well-known annealing softening phenomenon.

The herein disclosed manufacturing process may include extrusion alone, i.e. without post-extrusion annealing. In such extrusions, the combination of extrusion parameters of temperature, speed and ratio is such that the as-extruded microstructure is fully recrystallised and has a strong basal texture and a relatively large grain size.

In embodiments, a zinc alloy that comprises not more than 1.0 wt. % total percentage of added alloying elements, including iron, copper, calcium, magnesium, manganese, and lithium, the balance as zinc and unavoidable impurities, is prepared by casting.

In embodiments, the as-cast alloy is homogenized at about 350 °C for 2 hours, following by hot extrusion. To obtain a strong basal texture and a fully recrystallised microstructure in the as-extruded state, the extrusion temperature is controlled above 175 °C, and extrusion ratio is controlled to be in the range of 19:1-50:1.

Subsequently, the extrudate is subjected to an optional post-extrusion annealing treatment at a temperature ranging from about 250 °C to about 400 °C. Compared with the as-extruded samples, samples subjected to post-extrusion annealing exhibit a higher compressive yield strength, with their compressive yield strength increasing with an increase in annealing temperature, and improved creep resistance at human body temperature.

It is known that strength and other mechanical properties of a zinc alloy are highly dependent on the alloy microstructure and texture. The present inventors have discovered that only zinc-alloy samples with a strong basal texture exhibit the phenomenon of annealing strengthening, while non-basal textured zinc-alloy samples exhibit the conventional annealing softening behaviour in the uniaxial compression deformation at room temperature. Without being bound by theory, the reason why a zinc alloy having a strong basal texture is strengthened by annealing is that, in the deformation of the alloy, the loading direction is nearly parallel to basal planes of most grains, hence the most readily activated and operated basal slip is inhibited. Due to a high homologous temperature of zinc at room temperature, i.e., T/Tₘ=0.4, creep based intergranular deformation can occur instead, during plastic deformation at room temperature. However, the ease of intergranular deformation decreases with an increase in grain size, and non-basal slip modes having higher activation stresses have to activate and operate in coarse-grained samples to contribute plastic strain. Hence, the compressive strength of annealed samples that have relatively large grains is higher than that of as-extruded ones that have relatively smaller grains. In contrast, in the compressive deformation of non-basal textured zinc-alloy samples, basal slip is dominant in all samples, irrespectively of grain sizes, and their compressive yield strength decreases with an increase in grain size.

The microstructure and texture of the zinc-alloy can be effectively modified by applying hot extrusion under various conditions. Herein, extrusion temperature is a major factor that influences texture and recrystallisation fraction in the extruded zinc-alloy. For instance, when dilute zinc alloys are extruded at 175 °C or below, as-extruded samples exhibit a partial-recrystallized microstructure and a weak non-basal texture, and the non-basal texture is strengthened by post-extrusion annealing of the extruded samples. In contrast, with extrusion temperature increased to above 175 °C, as-extruded samples have a fully recrystallised microstructure and a strong basal texture, and the basal texture is retained after post-extrusion annealing.

In embodiments, the extrusion is performed at a temperature above 175 °C, or above about 180 °C, or above about 190 °C, or above about 200 °C, or above about 210 °C, or above about 220 °C, or above about 230 °C, or above about 240 °C.

In embodiments, the extrusion is performed at a temperature from about 180 °C to about 380 °C, or from about 180 °C to about 350 °C, or from about 180 °C to about 300 °C, or from about 180 °C to about 270 °C, or from about 180 °C to about 260 °C, or from about 180 °C to about 250 °C, or from about 200 °C to about 280 °C, or from about 200 °C to about 270 °C, or from about 200 °C to about 260 °C, or from about 200 °C to about 250 °C.

In embodiments, the extrusion speed is from about 0.01 mm·s⁻¹ to about 10 mm·s⁻¹, or from about 0.05 mm·s⁻¹ to about 5 mm·s⁻¹, or from about 0.05 mm·s⁻¹ to about 1 mm·s⁻¹, or from about 0.05 mm·s⁻¹ to about 0.5 mm·s⁻¹, or from about 0.05 mm·s⁻¹ to about 0.4 mm·s⁻¹, or from about 0.05 mm·s⁻¹ to about 0.3 mm·s⁻¹, or from about 0.05 mm·s⁻¹ to about 0.2 mm·s⁻¹.

In embodiments, the extrusion ratio is from about 8:1 to about 80:1, or from about 19:1 to about 60:1, or from about 19:1 to about 50:1, or from about 19:1 to about 45:1, or from about 25:1 to about 45:1, or from about 30:1 to about 40:1.

In embodiments, the annealing is performed at a temperature from about 250 °C to about 400 °C, or from about 250 °C to about 375 °C, or from about 275 °C to about 400 °C, or from about 275 °C to about 375 °C.

In embodiments, the annealing is performed from about 0.5 hours to about 50 hours, or from about 1 hour to about 10 hours, or from about 1 hour to about 5 hours.

In embodiments, an average grain size of the post-extruded alloy is greater than about 5 micron, or greater than about 6 micron, or greater than about 7 micron, or greater than about 8 micron, or greater than about 9 micron, or greater than about 10 micron.

In embodiments, an average grain size of the post-extruded alloy is less than about 25 micron, or from about 5 micron to about 25 micron

In embodiments, the annealing process increases an average grain size of the alloy.

In embodiments, an average grain size of the post-annealed alloy is greater than 25 about micron, or greater than about 30 micron, or greater than about 40 micron, or greater than about 50 micron, or greater than about 60 micron, or greater than about 70 micron, or greater than about 80 micron.

In embodiments, an average grain size of the post-annealed alloy is from about 25 micron to about 1000 micron, or from about 25 micron to about 1000 micron, or from about 25 micron to about 800 micron, or from about 25 micron to about 600 micron, or from about 25 micron to about 400 micron, or from about 25 micron to about 200 micron.

In a particular embodiment, the selected alloying elements are added in dilute amounts (at or less than 1.0 wt. %) and the extrusion parameters such as temperature, ratio and speed are such that extruded samples have a strong basal texture and a fully recrystallised microstructure. The extrusion temperature is controlled above 175 °C, the extrusion ratio is controlled to be in the range of 19:1-50:1 to obtain extruded samples having a strong basal texture and a fully recrystallised microstructure. The extrusion pushing rod has a relatively low speed, e.g., 0.05 mm·s⁻¹ to 0.5 mm·s⁻¹ to obtain a good surface finish of extruded rods.

In embodiments, the annealing process increases the alloy's ambient temperature compressive strength.

In embodiments, the annealing process decreases the alloy's minimum creep rate. In some embodiments, the annealing process decreases the alloy's minimum creep rate by a factor of 2, or a factor of 3, or a factor of 4, or a factor of 5, or a factor of 6, or a factor of 7, or a factor of 8, or a factor of 9, or a factor of 10.

### Alloy Composition

The dilute zinc alloy of the present disclosure comprises at least 99.0 wt. % zinc;
one or both of iron and copper in total amount from about 0.01 wt. % to about 0.99 wt. %;
from about 0.01 wt. % to about 0.7 wt. % of one or more of magnesium, calcium, manganese, and lithium; and
the sum of iron, copper, and one or more of magnesium, calcium, manganese, and lithium does not exceed about 1.0 wt. %.

In embodiments, the dilute zinc alloy comprises at least 99.1 wt. % zinc, or at least 99.2 wt. % zinc, or at least 99.3 wt. % zinc, or at least 99.4 wt. % zinc, or at least 99.5 wt. % zinc, or at least 99.6 wt. % zinc, or at least 99.7 wt. % zinc.

In some embodiments, the dilute zinc alloy comprises from about 0.01 wt. % to about 0.90 wt. % iron, or from about 0.01 wt. % to about 0.80 wt. % iron, or from about 0.01 wt. % to about 0.70 wt. % iron, or from about 0.01 wt. % to about 0.60 wt. % iron, or from about 0.01 wt. % to about 0.50 wt. % iron, or from about 0.01 wt. % to about 0.40 wt. % iron, or from about 0.01 wt. % to about 0.30 wt. % iron, or from about 0.01 wt. % to about 0.20 wt. % iron, or from about 0.01 wt. % to about 0.10 wt. % iron, or from about 0.01 wt. % to about 0.05 wt. % iron.

In some embodiments, the dilute zinc alloy comprises from about 0.01 wt. % to about 0.90 wt. % copper, or from about 0.01 wt. % to about 0.80 wt. % copper, or from about 0.01 wt. % to about 0.70 wt. % copper, or from about 0.01 wt. % to about 0.60 wt. % copper, or from about 0.01 wt. % to about 0.50 wt. % copper, or from about 0.01 wt. % to about 0.40 wt. % copper, or from about 0.01 wt. % to about 0.30 wt. % copper, or from about 0.01 wt. % to about 0.20 wt. % copper, or from about 0.01 wt. % to about 0.10 wt. % copper, or from about 0.01 wt. % to about 0.05 wt. % copper.

In some embodiments, the dilute zinc alloy comprises from about 0.01 wt. % to about 0.90 wt. % one or both iron and copper, or from about 0.01 wt. % to about 0.80 wt. % one or both iron and copper, or from about 0.01 wt. % to about 0.70 wt. % one or both iron and copper, or from about 0.01 wt. % to about 0.60 wt. % one or both iron and copper, or from about 0.01 wt. % to about 0.50 wt. % one or both iron and copper, or from about 0.01 wt. % to about 0.40 wt. % one or both iron and copper, or from about 0.01 wt. % to about 0.30 wt. % one or both iron and copper, or from about 0.01 wt. % to about 0.20 wt. % one or both iron and copper, or from about 0.01 wt. % to about 0.10 wt. % one or both iron and copper, or from about 0.01 wt. % to about 0.05 wt. % one or both iron and copper.

In some preferred embodiments, the dilute zinc alloy comprises from about 0.02 wt. % to about 0.60 wt. % one or both iron and copper.

In some embodiments, the dilute zinc alloy comprises from about 0.01 wt. % to about 0.70 wt. % of one or more of magnesium, calcium, manganese, and lithium, or from about 0.01 wt. % to about 0.60 wt. %, or from about 0.01 wt. % to about 0.50 wt. %, or from about 0.01 wt. % to about 0.40 wt. %, or from about 0.02 wt. % to about 0.50 wt. %, or from about 0.02 wt. % to about 0.40 wt. %.

In embodiments, the sum of iron, copper, magnesium, calcium, manganese, and lithium in the dilute zinc alloy is from about 0.05 wt. % to about 1.0 wt. %, or from about 0.05 wt. % to about 0.9 wt. %, or from about 0.05 wt. % to about 0.8 wt. %, or from about 0.05 wt. % to about 0.7 wt. %.

In some preferred embodiments, the dilute zinc alloy comprises from about 99.1 wt. % to about 99.95 wt. % zinc, from about 0.01 wt. % to about 0.90 wt. % of one or both iron and copper, and from about 0.01 wt. % to about 0.70 wt. % of one or more of magnesium, calcium, manganese, and lithium.

In embodiments the compressive yield strength of the alloys is greater than 220 MPa when measured at ambient temperature and a strain rate of 10⁻³ s⁻¹, or greater than 230 MPa, or greater than 240 MP a, or greater than 250 MP a, or greater than 260 MPa, or greater than 270 MPa, or greater than 280 MPa, or greater than 290 MPa, or greater than 300 MP a.

In embodiments, the minimum compressive creep rate of the alloys is less than 3×10⁻⁶ s⁻¹ under a loading stress of 200 MPa at 37 °C, or less than 2×10⁻⁶ s⁻¹, or less than 1×10⁻⁶ s⁻¹, or less than 5×10⁻⁷ s⁻¹, or less than 3×10⁻⁷ s⁻¹, or less than 2×10⁻⁷ s⁻¹, or less than 1×10⁻⁷ s⁻¹.

### EXAMPLES

### Analysis Techniques

The compositions of metal alloys were determined by inductively coupled plasma atomic emission spectroscopy (ICP-AES).

EBSD characterisation was conducted on a JEOL JSM-7001F FEG SEM, equipped with a Nordlys Max² EBSD detector. Oxford Instruments Aztec software as the acquisition system was used to control the data processing, and post data analyses, including EBSD orientation mapping, grain size measurement, and inverse pole figure contouring, were conducted on Channel 5 HKL software.

Uniaxial compressive and tensile tests were conducted using an Instron 5982 machine that had a fixed load cell of 100 kN and was controlled with Bluehill®2 software. Cylindrical compression specimens with 6 mm in diameter and 9 mm in height were machined along the ED, with a standard height to diameter ratio of 1.5:1, according to the ASTM E9-09 (2009) standard. The compressive tests were conducted with a crosshead speed of 0.54 mm min⁻¹, corresponding to a strain rate of 10⁻³ s⁻¹. Round dog-shaped tensile specimens with 15 mm gauge length and 3.5 mm gauge diameter were machined along the ED for tensile tests. The dimensions of tensile specimens followed the ASTM E8M (2013) standard. Tensile tests were conducted with a clip-on extensometer with 10 mm in gauge length, and the crosshead speed was 0.9 mm min⁻¹, corresponding to a strain rate of 10⁻³ s⁻¹.

Constant-load compressive creep tests were conducted on an Instron 5982 machine equipped with two hairdryers for temperature control, and a thermometer was set to measure the specimen surface temperature. Cylindrical compression specimens of 6 mm diameter and 9 mm height were machined along the ED, with a standard height to diameter ratio of 1.5:1, according to the ASTM E9-09 (2009) standard. Before creep testing, specimens were heated at 37 °C for 10 minutes, and the temperature fluctuated within ±1 °C. During creep testing, specimens were tested under a constant stress of 200 MPa or 250 MP a until manually stopped after 2 hours. The creep data were collected using Bluehill®2 software.

### Alloy Preparation and Processing

The purities of the metals were: Zn (99.95%), Fe (99.98%), Cu (99.99%), Ca (99%), and Mg (99.95%).

The dilute zinc alloys were prepared by induction melting in a graphite crucible at approximately 550 °C under an argon atmosphere. The molten alloys were then cast into a pre-heated steel mould (~150 °C) coated with boron nitride, followed by natural cooling to room temperature. The as-cast ingots were cylinders about 1 kg in weight and with a size of 38 mm in diameter and 140 mm in length. Cylindrical billets with dimensions of φ35×60 mm³ were machined from the as-cast ingots for subsequent homogenization, hot extrusion, and post-extrusion annealing treatments. Table 1 summarises the alloy compositions. Prior to hot extrusion, the machined cast billets were homogenised by heat-treatments at 300 °C for 2 hours in a muffle furnace, followed by water quenching, and then were extruded into cylindrical rods of 6 mm in diameter with a ram speed of 0.1 mm s⁻¹, corresponding to an extrusion ratio of about 36:1. During extrusion processes, all billets were preheated at their extrusion temperatures for 10 minutes and extrusions were directly quenched into room temperature water. Post-extrusion annealing heat treatments were conducted in a muffle furnace, followed by water quenching.

TABLE 1
(measured wt. %; the balance is Zn
and other trace impurity elements)

| Designation | Fe | Cu | Ca | Mg | Mn |
|---|---|---|---|---|---|
| M3 | 0.01 | 0.04 | 0.00 | 0.05 | 0.00 |
| M4 | 0.00 | 0.06 | 0.00 | 0.04 | 0.00 |
| M5 | 0.06 | 0.14 | 0.01 | 0.16 | 0.09 |
| M6 | 0.01 | 0.37 | 0.00 | 0.18 | 0.05 |
| M7 | 0.02 | 0.10 | 0.02 | 0.15 | 0.16 |

### Example 1: Annealing Strengthening of M3 Alloy

As-cast M3 alloy was homogenized at 300 °C for 2 hours, followed by water quenching. The homogenized billet was extruded into a cylindrical bar of 6 mm diameter at a temperature above 175 °C with a pushing rod speed of 0.1 mm·s⁻¹. The extrusion ratio was controlled to be about 36:1. After hot extrusion, annealing treatments were conducted at temperatures of 275 °C, 300 °C, and 350 °C for 2 hours. Table 2 summarises the conditions of extrusion and post-extrusion annealing (if performed).

TABLE 2

| | Designation | | | |
|---|---|---|---|---|
| | Extrusion | | Post-extrusion annealing | |
| | Temperature | Extrusion | Temperature | Time |
| M3 | (°C) | ratio | (°C) | (h) |
| 250E | 250 | 36:1 | — | — |
| 250E + 275 °C/2 h | 250 | 36:1 | 275 | 2 |
| 250E + 300 °C/2 h | 250 | 36:1 | 300 | 2 |
| 250E + 350 °C/2 h | 250 | 36:1 | 350 | 2 |

The various extrusion and post-extrusion annealing conditions produced different grain sizes ranging from 11 to 95 μm, as shown in Table 3.

TABLE 3

| | Designation | | | |
|---|---|---|---|---|
| | Average | Compressive | Tensile | Minimum |
| | grain size | YS | YS | compressive |
| M3 | (μm) | (MPa) | (MPa) | creep rate (s⁻¹) |
| 250E | 11 | 277 | 247 | 2.4 × 10⁻⁶ |
| 250E + 275 °C/2 h | 27 | 285 | — | — |
| 250E + 300 °C/2 h | 36 | 290 | 206 | — |
| 250E + 350 °C/2 h | 95 | 305 | — | 2.5 × 10⁻⁷ |

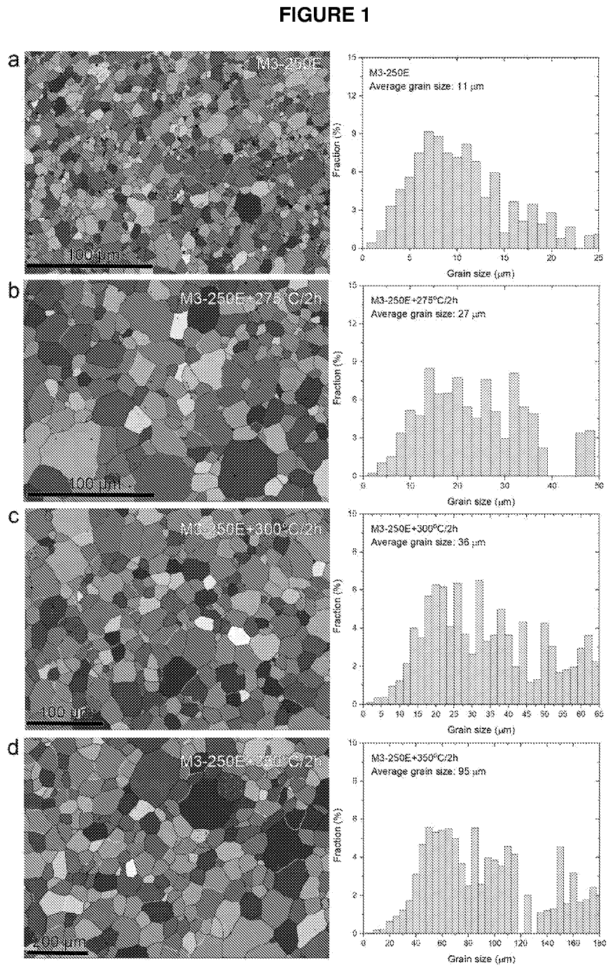

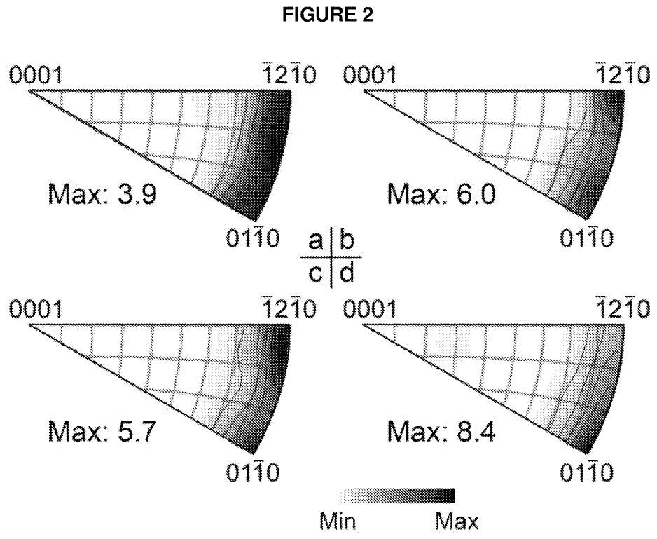

FIG. 1 shows the microstructures (left hand images) and grain size distributions (right hand images) of M3 alloys, respectively (a) 250E, (b) 250E+275 °C/2 h, (c) 250E+300 °C/2 h, and (d) 250E+350 °C/2 h. FIG. 2 shows the textures of the same four samples.

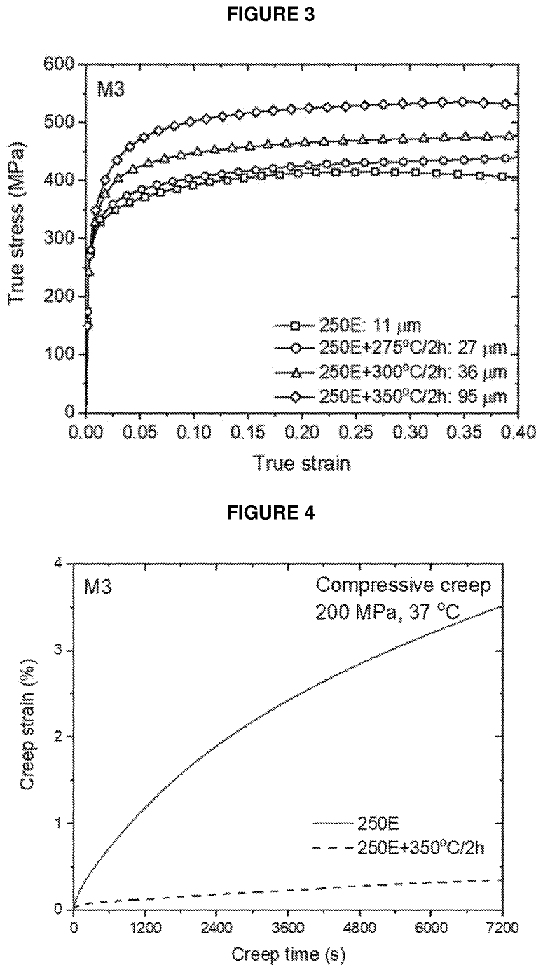

As-extruded and annealed samples were subjected to uniaxial compressive deformation along the extrusion direction at room temperature and a strain rate of 10⁻³ s⁻¹. The true stress-strain curves are shown in FIG. 3 and the compressive yield strength values are listed in Table 3. The as-extruded alloy (250E) had a compressive yield strength of 277 MPa, which is lower than all annealed alloys, which had their compressive strengths increased with an increase in annealing temperature, which is 285 MP a for the 250E+275 °C/2 h sample, 290 MP a for the 250E+300 °C/2 h sample, and 305 MP a for the 250E+350 °C/2 h sample. The post-extrusion annealing at 350 °C for 2 hours provided the greatest strength improvement of the extruded M3 alloy. The compressive creep tests for M3 alloy extruded at 250 °C and a subsequent annealing treatment at 350 °C for 2 hours were examined under a loading stress of 200 MPa at 37 °C. The creep curves are shown in FIG. 4, and the minimum creep rates are listed in Table 3. For M3 alloy extruded at 250 °C, the sample annealed at 350 °C for 2 hours had the minimum creep rate (2.5×10⁻⁷ s⁻¹) which is ten-fold lower than the as-extruded sample, indicating that the post-extrusion annealing treatment effectively improves the creep resistance of the M3 alloy.

### Example 2: Annealing Strengthening of M4 Alloy

As-cast M4 alloy was homogenized at 300 °C for 2 hours, followed by water quenching. The homogenized billets were extruded into a cylindrical bar of 6 mm diameter at a temperature above 200 °C with a pushing rod speed of 0.1 mm·s⁻¹. The extrusion ratio was controlled to be about 36:1. After hot extrusion, annealing treatments were conducted at temperatures of 215 °C, 300 °C, and 350 °C for 2 hours. Table 4 summarises the conditions of extrusion and post-extrusion annealing (if performed).

TABLE 4

| | Designation | | | |
|---|---|---|---|---|
| | Extrusion | | Post-extrusion annealing | |
| | Temperature | Extrusion | Temperature | Time |
| M4 | (°C) | ratio | (°C) | (h) |
| 250E | 250 | 36:1 | — | — |
| 250E + 275 °C/2 h | 250 | 36:1 | 275 | 2 |
| 250E + 300 °C/2 h | 250 | 36:1 | 300 | 2 |
| 250E + 350 °C/2 h | 250 | 36:1 | 350 | 2 |

As-extruded rods had a fully recrystallised microstructure and a strong basal texture. The various extrusion and post-extrusion annealing conditions produced different grain sizes ranging from about 22 to about 630 μm, as shown in Table 5.

TABLE 5

| | Designation | | | |
|---|---|---|---|---|
| | Average | Compressive | Tensile | Minimum |
| | grain size | YS | YS | compressive |
| M4 | (μm) | (MPa) | (MPa) | creep rate (s⁻¹) |
| 250E | 22 | 250 | 218 | 1.9 × 10⁻⁶ |
| 250E + 275 °C/2 h | 55 | 277 | — | — |
| 250E + 300 °C/2 h | 88 | 308 | 139 | — |
| 250E + 350 °C/2 h | 630 | 321 | — | 1.9 × 10⁻⁷ |

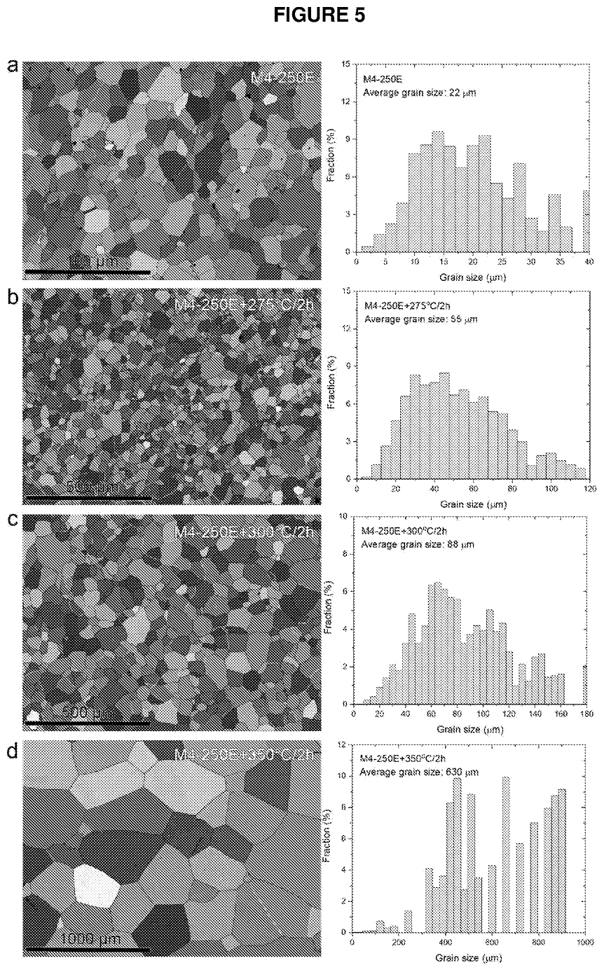

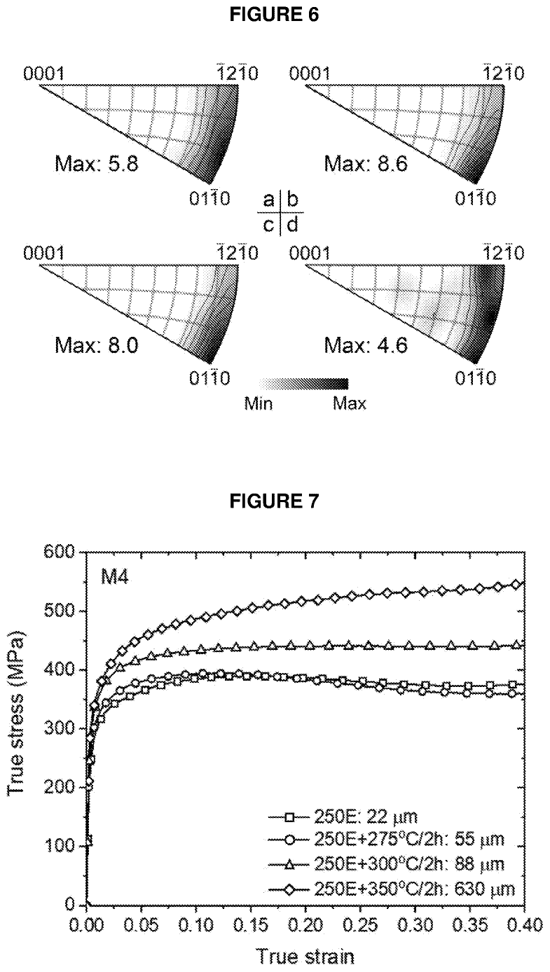

FIG. 5 shows the microstructures (left hand images) and grain size distributions (right hand images) of M4 alloys, respectively (a) 250E, (b) 250E+275 °C/2 h, (c) 250E+300 °C/2 h, and (d) 250E+350 °C/2 h. The basal texture was retained after the annealing treatments, as shown in the corresponding inverse pole figures in FIG. 6.

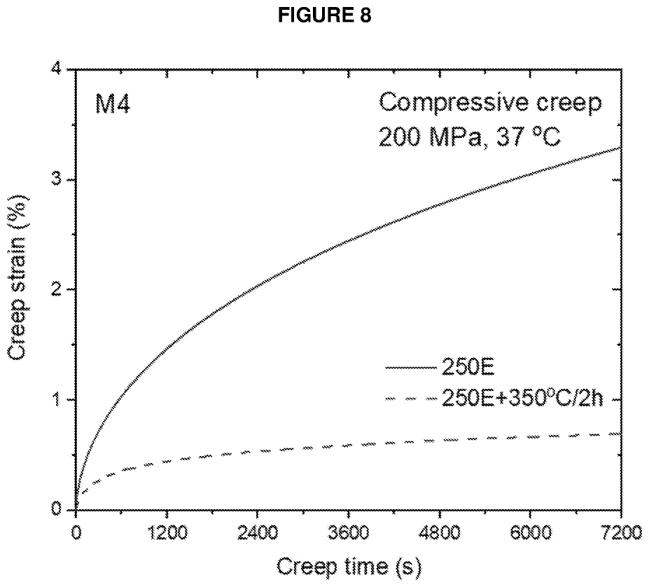

Uniaxial compressive deformation was conducted along the extrusion direction at room temperature and a strain rate of 10⁻³ s⁻¹. The obtained true stress-strain curves are shown in FIG. 7, and the compressive yield strength values of the extruded and annealed M4 alloy samples are listed in Table 5. The 250 °C extruded alloy sample with an average grain size of about 22 μm had the lowest compressive yield strength of 250 MPa. With an increase in grain size, the compressive yield strengths of the samples of 250E+275 °C/5 h (55 μm), 250E+300 °C/2 h (88 μm), and 250E+350 °C/24 h (630 μm) were 277 MPa, 308 MPa, and 321 MPa, respectively. The compressive creep tests of the M4 alloy samples in the 250E and 250E+350 °C/2 h conditions were examined under a loading stress of 200 MPa at 37 °C (human body temperature). The creep curves are shown in FIG. 8 and the minimum creep rates are listed in Table 5. The application of post-extrusion annealing at 350 °C for 2 hours improved the creep resistance of the M4 alloy extruded at 250 °C by about one order of magnitude.

### Example 3: Effect of Loading Direction on Annealing Strengthening Response of the M3 and M4 Alloy

The effect of loading direction on annealing strengthening response of M3 and M4 alloys was investigated. The uniaxial tensile deformation of M3 and M4 alloy subjected to extrusion at 250 °C and a subsequent annealing at 300 °C for 2 hours was conducted at room temperature and a strain rate of 10⁻³ s⁻¹, and the loading direction was parallel to the extrusion direction. Tables 3 and 5 list the tensile yield strength values (Tensile YS). For both M3 and M4 alloys subjected to extrusion at 250 °C, the annealing strengthening response that was observed in the uniaxial compression tests was absent in the tensile deformation.

### Example 4: Annealing Strength of M5 Alloy

As-cast M5 alloy was homogenized at 300 °C for 2 hours, followed by water quenching. The homogenized billets were extruded into a cylindrical bar of 6 mm diameter at a temperature above 200 °C with a pushing rod speed of 0.1 mm·s⁻¹. The extrusion ratio was controlled to be about 36:1. After hot extrusion, annealing treatments were conducted at a temperature of 350 °C for 2 hours. Table 6 summarises the conditions of extrusion and post-extrusion annealing (if performed).

TABLE 6

| | Designation | | | |
|---|---|---|---|---|
| | Extrusion | | Post-extrusion annealing | |
| | Temperature | Extrusion | Temperature | Time |
| M5 | (°C) | ratio | (°C) | (h) |
| 250E | 250 | 36:1 | — | — |
| 250E + 350 °C/2 h | 250 | 36:1 | 350 | 2 |
| 300E | 300 | 36:1 | — | — |
| 300E + 350 °C/2 h | 300 | 36:1 | 350 | 2 |

As-extruded rods had a fully recrystallised microstructure and a strong basal texture. The various extrusion and post-extrusion annealing conditions produced different grain sizes ranging from about 12 to about 129 μm, as shown in Table 7.

TABLE 7

| | Designation | | | |
|---|---|---|---|---|
| | Average | Compressive | Tensile | Minimum |
| | grain size | YS | YS | compressive |
| M5 | (μm) | (MPa) | (MPa) | creep rate (s⁻¹) |
| 250E | 12 | 350 | 294 | 1.4 × 10⁻⁶ |
| 250E + 350 °C/2 h | 32 | 360 | 255 | 1.2 × 10⁻⁷ |
| 300E | 16 | 359 | — | — |
| 300E + 350 °C/2 h | 129 | 381 | — | — |

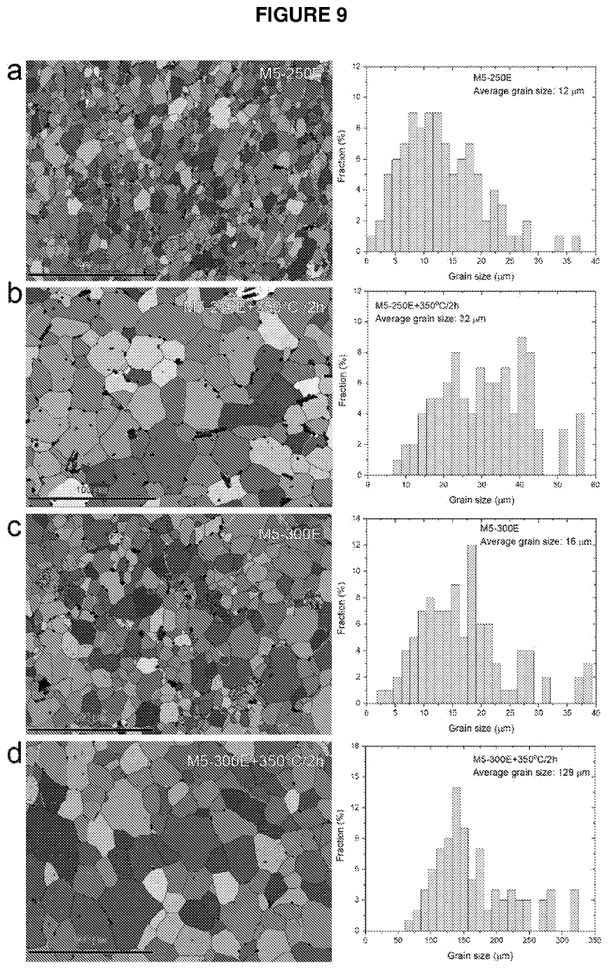

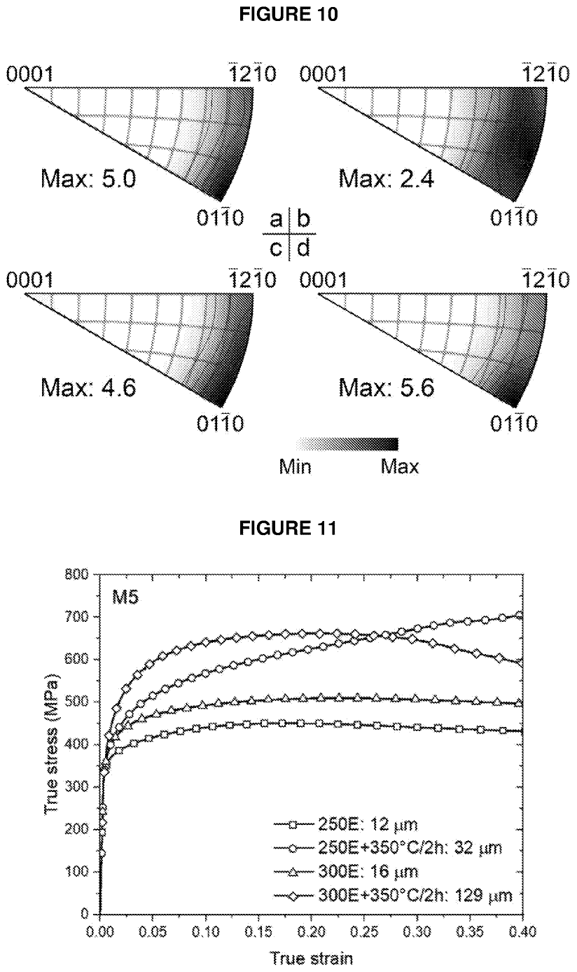

FIG. 9 shows the microstructures (left hand images) and grain size distributions (right hand images) of M5 alloys, respectively (a) 250E, (b) 250E+350 °C/2 h, (c) 300E, and (d) 300E+350 °C/2 h. The basal texture was formed during the extrusion process and retained after the annealing treatments, as shown in the corresponding inverse pole figures in FIG. 10.

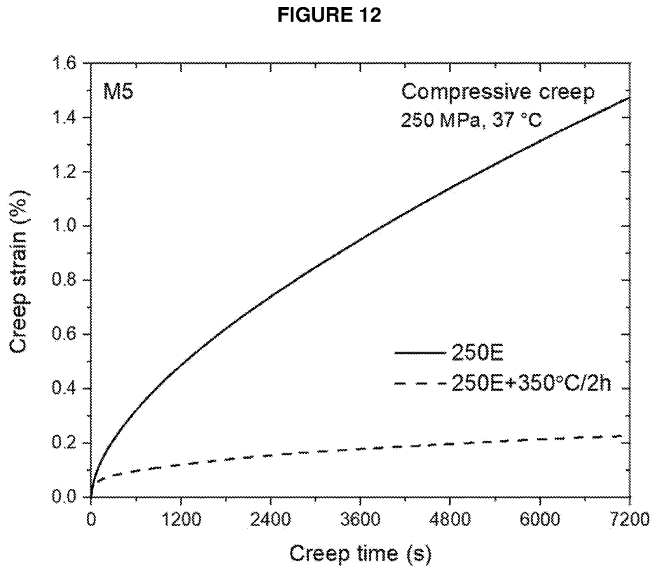

Uniaxial compressive deformation was conducted along the extrusion direction at room temperature and a strain rate of 10⁻³ s⁻¹. The obtained true stress-strain curves are shown in FIG. 11, and the compressive yield strength values of the extruded and annealed M5 alloy samples are listed in Table 7. The 250 °C extruded alloy sample with an average grain size of about 12 μm had the lowest compressive yield strength of 350 MPa. With an increase in grain size, the compressive yield strengths of the samples of 300E (16 μm), 250E+350 °C/2 h (32 μm), and 300E+350 °C/2 h (129 μm) were 359 MPa, 360 MPa, and 381 MPa, respectively. The compressive creep tests of the M5 alloy samples in the 250E and 250E+350 °C/2 h conditions were examined under a loading stress of 250 MP a at 37 °C (human body temperature). The creep curves are shown in FIG. 12 and the minimum creep rates are listed in Table 7. The application of post-extrusion annealing at 350 °C for 2 hours improved the creep resistance of the M5 alloy extruded at 250 °C by more than one order of magnitude.

### Example 5: Annealing Strength of M6 Alloy

As-cast M6 alloy was homogenized at 300 °C for 2 hours, followed by water quenching. The homogenized billets were extruded into a cylindrical bar of 6 mm diameter at a temperature above 200 °C with a pushing rod speed of 0.1 mm·s⁻¹. The extrusion ratio was controlled to be about 36:1. After hot extrusion, annealing treatments were conducted at a temperature of 350 °C for 2 hours and 6 hours. Table 8 summarises the conditions of extrusion and post-extrusion annealing (if performed).

TABLE 8

| | Designation | | | |
|---|---|---|---|---|
| | Extrusion | | Post-extrusion annealing | |
| | Temperature | Extrusion | Temperature | Time |
| M6 | (°C) | ratio | (°C) | (h) |
| 250E | 250 | 36:1 | — | — |
| 250E + 350 °C/6 h | 250 | 36:1 | 350 | 6 |
| 300E | 300 | 36:1 | — | — |
| 300E + 350 °C/2 h | 300 | 36:1 | 350 | 2 |

As-extruded rods had a fully recrystallised microstructure and a strong basal texture. The various extrusion and post-extrusion annealing conditions produced different grain sizes ranging from about 8 to about 208 μm, as shown in Table 9.

TABLE 9

| | Designation | | | |
|---|---|---|---|---|
| | Average | Compressive | Tensile | Minimum |
| | grain size | YS | YS | compressive |
| M6 | (μm) | (MPa) | (MPa) | creep rate (s⁻¹) |
| 250E | 8 | 326 | 268 | 1.5 × 10⁻⁶ |
| 250E + 350 °C/6 h | 43 | 386 | — | — |
| 300E | 23 | 355 | 236 | — |
| 300E + 350 °C/2 h | 208 | 398 | — | 3.5 × 10⁻⁸ |

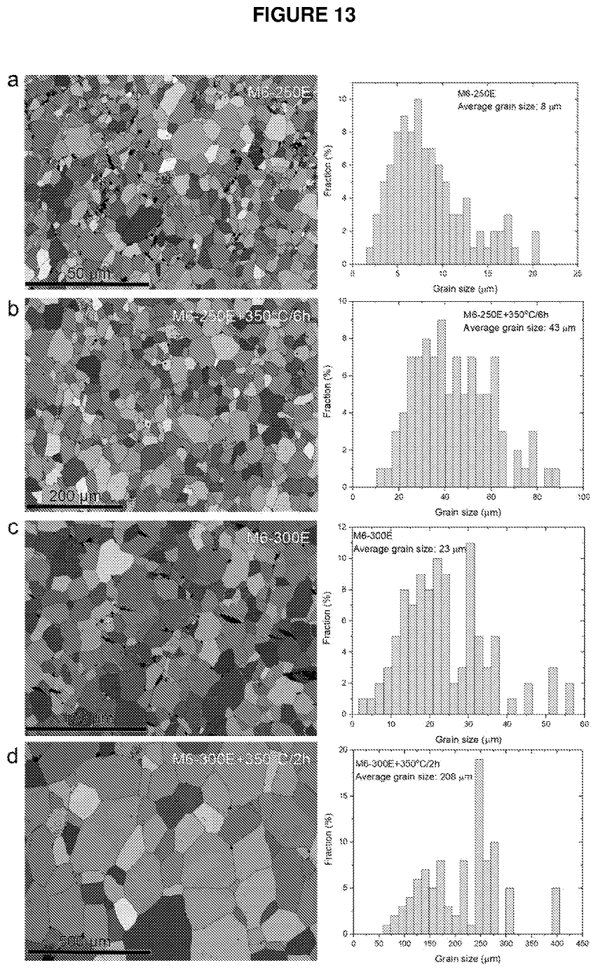

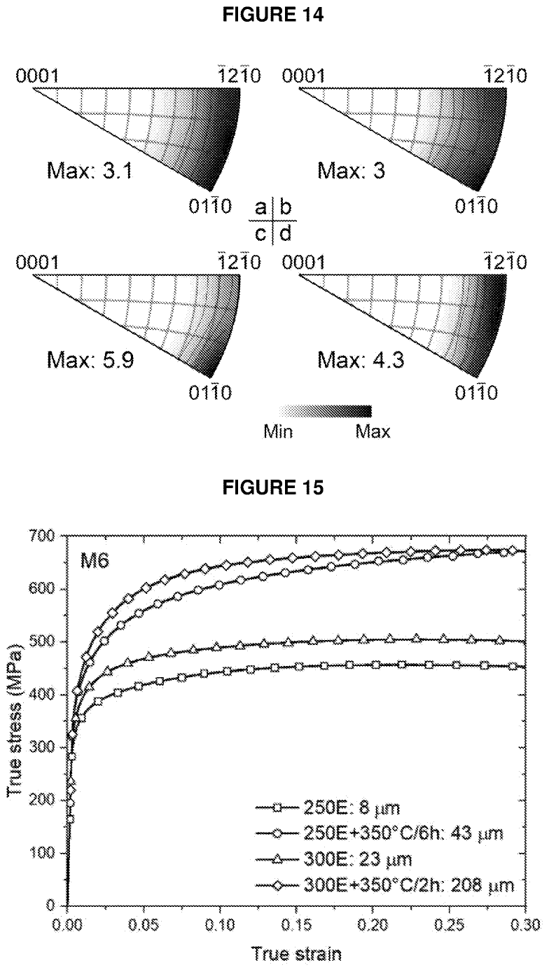

FIG. 13 shows the microstructures (left hand images) and grain size distributions (right hand images) of M6 alloys, respectively (a) 250E, (b) 250E+350 °C/6 h, (c) 300E, and (d) 300E+350 °C/2 h. The basal texture was formed during the extrusion process and retained after the annealing treatments, as shown in the corresponding inverse pole figures in FIG. 14.

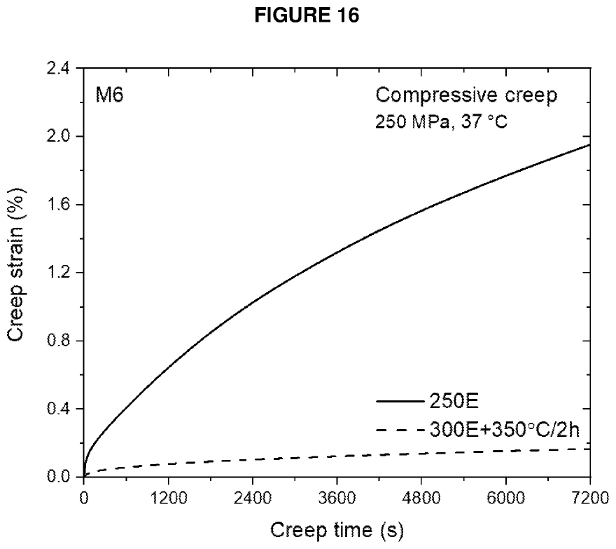

Uniaxial compressive deformation was conducted along the extrusion direction at room temperature and a strain rate of 10⁻³ s⁻¹. The obtained true stress-strain curves are shown in FIG. 15 and the compressive yield strength values of the extruded and annealed M6 alloy samples are listed in Table 9. The 250 °C extruded alloy sample with an average grain size of about 8 μm had the lowest compressive yield strength of 326 MPa. With an increase in grain size, the compressive yield strengths of the samples of 300E (23 μm), 250E+350 °C/6 h (43 μm), and 300E+350 °C/2 h (208 μm) were 355 MPa, 386 MPa, and 398 MPa, respectively. The compressive creep tests of the M6 alloy samples in the 250E and 300E+350 °C/2 h conditions were examined under a loading stress of 250 MP a at 37 °C (human body temperature). The creep curves are shown in FIG. 16 and the minimum creep rates are listed in Table 9. The M6 alloy extruded at 300 °C and then subjected to post-extrusion annealing at 350 °C for 2 hours exhibits a more than one order of magnitude improvement in creep resistance compared to the sample extruded at 250 °C.

### Example 6: Annealing Strength of M7 Alloy

As-cast M7 alloy was homogenized at 300 °C for 2 hours, followed by water quenching. The homogenized billets were extruded into a cylindrical bar of 6 mm diameter at a temperature above 300 °C with a pushing rod speed of 0.1 mm·s⁻¹. The extrusion ratio was controlled to be about 36:1. After hot extrusion, annealing treatments were conducted at temperatures of 350 °C and 370 °C for variable durations between 0.5 and 3 hours. Table 10 summarises the conditions of extrusion and post-extrusion annealing (if performed).

TABLE 10

| | Designation | | | |
|---|---|---|---|---|
| | Extrusion | | Post-extrusion annealing | |
| | Temper- | | Temper- | |
| | ature | Extrusion | ature | Time |
| M7 | (°C) | ratio | (°C) | (h) |
| 300E | 300 | 36:1 | — | — |
| 300E + 350 °C/0.5 h | 300 | 36:1 | 350 | 0.5 |
| 300E 350 °C/2 h | 300 | 36:1 | 350 | 2 |
| 300E + 370 °C/3 h | 300 | 36:1 | 370 | 3 |

As-extruded rods had a fully recrystallised microstructure and a strong basal texture. The various extrusion and post-extrusion annealing conditions produced different grain sizes ranging from about 21 to about 65 μm, as shown in Table 11.

TABLE 11

| | Designation | | | |
|---|---|---|---|---|
| | Average | Com- | | Minimum |
| | grain | pressive | Tensile | comp- |
| | size | YS | YS | ressive |
| M7 | (μm) | (MPa) | (MPa) | creep rate (s⁻¹) |
| 300E | 21 | 365 | 257 | 4.7 × 10⁻⁷ |
| 300E + 350 °C/0.5 h | 28 | 394 | 234 | — |
| 300E + 350 °C/2 h | 32 | 406 | — | — |
| 300E + 370 °C/3 h | 65 | 433 | — | 3.5 × 10⁻⁸ |

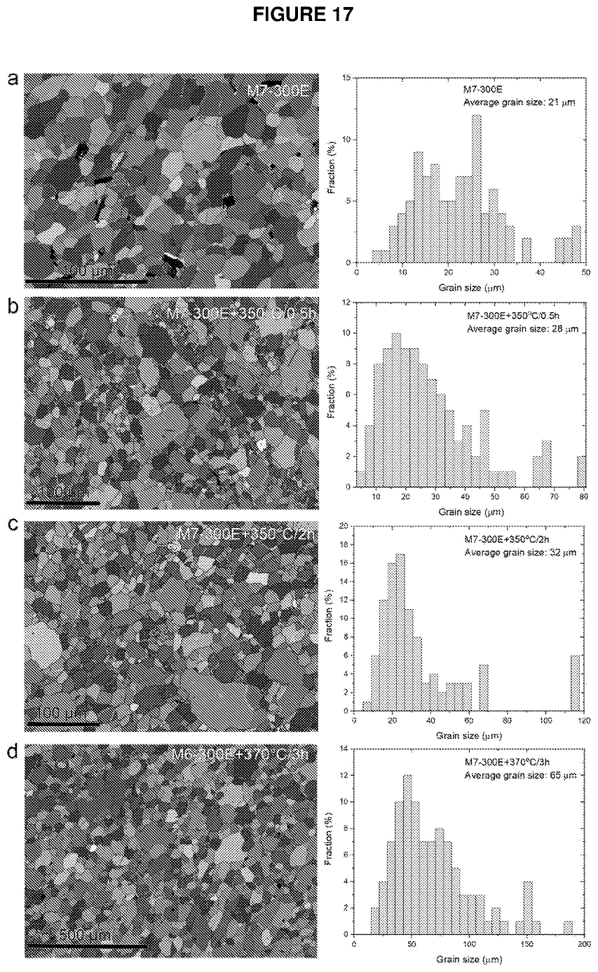

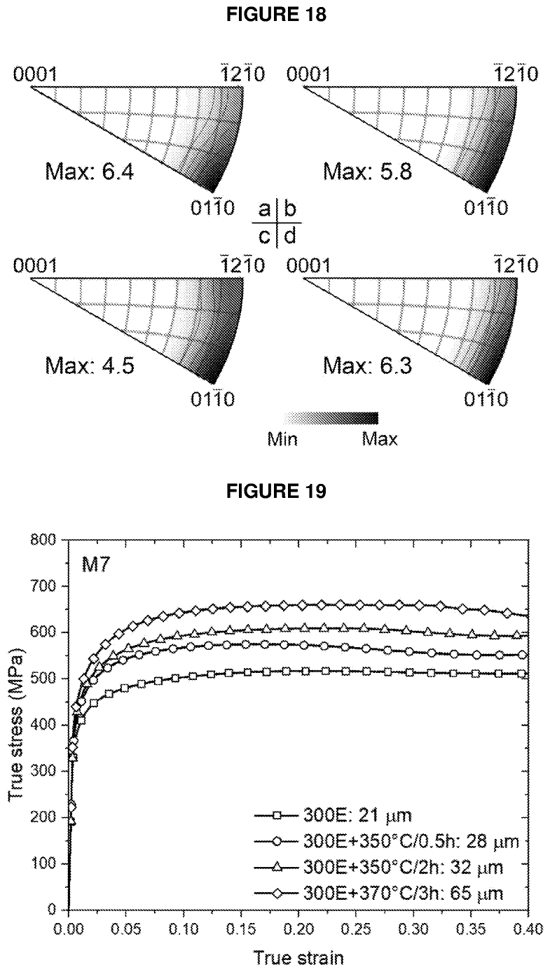

FIG. 17 shows the microstructures (left hand images) and grain size distributions (right hand images) of M7 alloys, respectively (a) 300E, (b) 300E+350 °C/0.5 h, (c) 300E+350 °C/2 h, and (d) 300E+370 °C/3 h. The basal texture was retained after the annealing treatments, as shown in the corresponding inverse pole figures in FIG. 18.

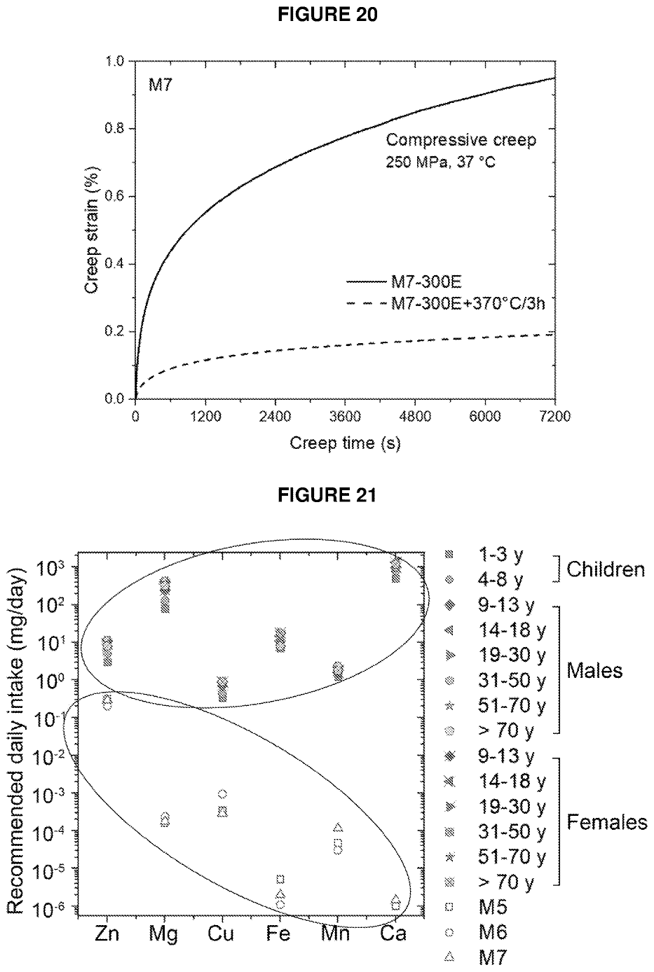

Uniaxial compressive deformation was conducted along the extrusion direction at room temperature and a strain rate of 10⁻³ s⁻¹. The obtained true stress-strain curves are shown in FIG. 19, and the compressive yield strength values of the extruded and annealed M7 alloy samples are listed in Table 11. The 300 °C extruded alloy sample with an average grain size of about 21 μm had the lowest compressive yield strength of 365 MPa. With an increase in grain size, the compressive yield strengths of the samples of 300E+350 °C/0.5 h (28 μm), 300E+350 °C/2 h (32 μm), and 300E+370 °C/3 h (65 μm) were 394 MPa, 406 MPa, and 433 MPa, respectively. The compressive creep tests of the M7 alloy samples in the 300E and 300E+370 °C/3 h conditions were examined under a loading stress of 250 MPa at 37 °C (human body temperature). The creep curves are shown in FIG. 20 and the minimum creep rates are listed in Table 11. The application of post-extrusion annealing at 370 °C for 3 hours improved the creep resistance of the M7 alloy extruded at 300 °C by about one order of magnitude.

### Example 7: In-Vitro Biodegradation Testing

Biocorrosion rates of the zinc-alloy samples were determined by immersion tests. Samples of 6 mm diameter and 1 mm thick were grinded and polished by 1 μm diamond suspension, weighed, sonicated in ethanol and immersed in Dulbecco's modified eagle medium (DMEM, Gibco, ThermoFisher Scientific Australia) at 37 °C and under 5% CO₂ atmosphere for 30 days, with a solution volume to sample surface area (V/S) ratio of 0.4 mL/mm², as guided by ASTM-G31-72 (1972). DMEM solution was refreshed every 48 hours. Following the immersion tests, corroded samples were treated with a solution containing 200 g/L CrO₃ and 10 g/L AgNO₃ for 15 minutes to remove corrosion products. The biocorrosion rate of each sample was calculated by the weight loss measured after an immersion period of 30 days, according to the equation outlined in ASTM-G31-72 (1972): corrosion rate=(K×W)/(A×T×p), where K is a constant (8.76×10⁴ for the unit of mm/year), W is the weight loss in the unit of gram, A is the exposed sample surface area in the unit of cm², T is the time of exposure in the unit of hours, and p is the sample density in the unit of g/cm³.

FIG. 21 shows the estimated daily doses of six elements of Zn, Mg, Cu, Fe, Mn, and Ca released from the alloys M5, M6, and M7 in the in-vitro biodegradation tests, assuming an implant surface area of 400 mm² (data points contained within the lower oval). These estimated values are compared against recommended daily intake values of these six elements for different human groups (data points within the upper oval). It is evident that the estimated daily doses released from the alloys were orders of magnitude lower than the recommended daily intake values.

### Example 8: Cell Viability Studies

The MG-63 (ATCC® CRL-1427™) human osteosarcoma cell line, which remains the human pre-osteoblast form and has the behaviour similar to that of immature in vivo osteoblasts in the early differentiation stage, was used for indirect contact cell viability tests. These tests employed methylthiazol tetrazolium salt (MTS, Cell Titre 96© Aqueous One Solution Cell Proliferation Assay, Promega, Australia) assay. A control sample, and a dilute magnesium alloy (Mg-0.4Zn-0.1Ca) extruded at 400 °C (see Example 11), was used for the purpose of comparison. The sample sterilization procedure involved disc samples of approximately 1 mm thickness, which were polished by 1 μm diamond suspension and sonicated in ethanol for a minimum of 30 minutes, and then exposed to UV light for 20 minutes (10 minutes for each side). The samples were then placed in an antimicrobial solution of 1 vol. % penicillin/streptomycin (Gibco, ThermoFisher Scientific Australia) and 99 vol. % sterile phosphate-buffered saline (PBS) at 4 °C for 1 hour, followed by washing in sterile PBS on a plate shaker for five cycles.

The MG-63 cells were cultured in low glucose (1 g/L) DMEM supplemented with 1 vol. % penicillin/streptomycin and 10 vol. % foetal bovine serum (FBS, Gibco, ThermoFisher Scientific Australia) in an incubator at 37 °C and 5% CO₂ and 95% humidity. MG-63 cells in the passage range of P5-P10 were selected to conduct the indirect MTS assay.

Cytotoxicity assessment was made following ISO 10993-5 (2009 Jun. 1)/ISO 10993-12 (2012 Jul. 1). On day 0, cells were seeded in a 96-well plate at a density of 10⁴ cells/cm² and incubated for 24 hours to facilitate cell attachment. Extracts were prepared by submerging samples in the cell culture media, with a surface area to medium ratio of 1.25 mL/cm², in accordance with ISO 10993-12 (2012 Jul. 1), at 37 °C in an environment of 5% CO₂ and 95% humidity for 24 hours. On day 1, the cell culture media were replaced by 100 μL/well sample extracts at 8 different concentrations (100%, 50%, 25%, 12.5%, 6.25%, 3.125%, 1.56%, and 0.78%) under incubation for 24 hours. Fresh cell culture media and 10% dimethyl sulfoxide (DMSO, Invitrogen, US) were used as negative control and positive control, respectively. On day 2, the extracts were replaced with 100 μL/well mixed solution of MTS reagent and cell culture media (1:5 dilution), and the plates were incubated for approximately 3 hours.

Optical absorbance of formazan product was measured at 490 nm using a microplate reader (PerkinElmer, US), and cell viability was calculated based on the formula established in ISO 10993-5 (2009 Jun. 1):

$$viability \% = \frac{100 \times {OD}_{e}}{{OD}_{nc}} ,$$

where ODₑ is the mean value of the measured optical density of extracts of test samples, and OD_{nc} is the mean value of the measured optical density of the negative control group. The mean value of the measured optical density of cell culture media background was subtracted from ODₑ and OD_{nc} values. Triplicates were measured for each alloy in a MTS assay. MTS assays were repeated three times independently. The cell viability data were analysed by using a one-way analysis of variance (ANOVA) followed by Tukey post hoc testing. The two datasets with significance level p<0.05 were considered to be statistically different.

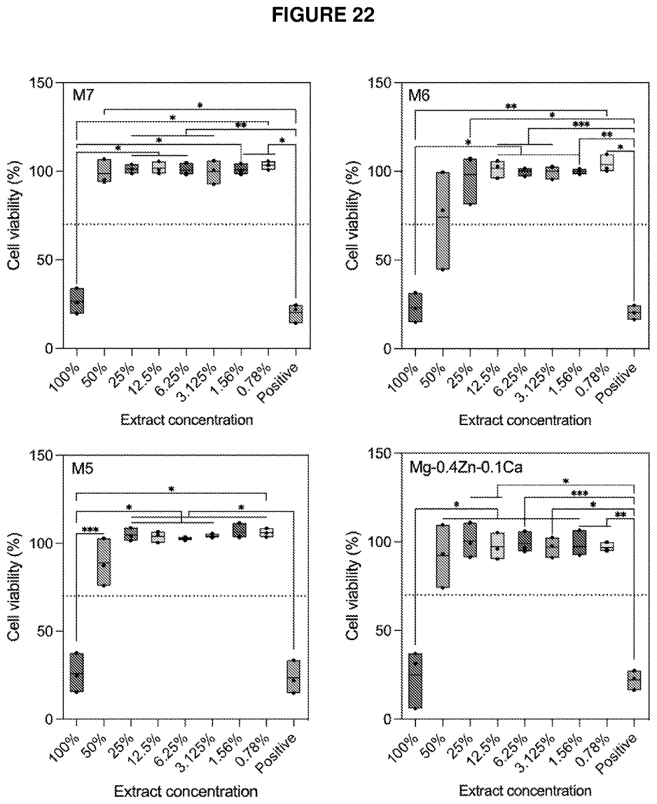

FIG. 22 shows the cell viability of MG-63 osteoblast cells exposed to extracts of M5, M6, and M7 alloys and the Mg-0.4Zn-0.1Ca alloy of Example 11, as the reference material, at 8 different concentrations for 24 hours. The dashed line across the middle of each plot indicates the threshold between toxic and non-toxic responses (70% cell viability), as per ISO 10993-5 (2009 Jun. 1). It is evident that, on the whole, non-toxic responses resulted when extracts of the dilute Zn alloys were diluted to 50% or lower, which is similar to the reference material (Mg-0.4Zn-0.1Ca).

### Example 9: Effect of Extrusion Temperature

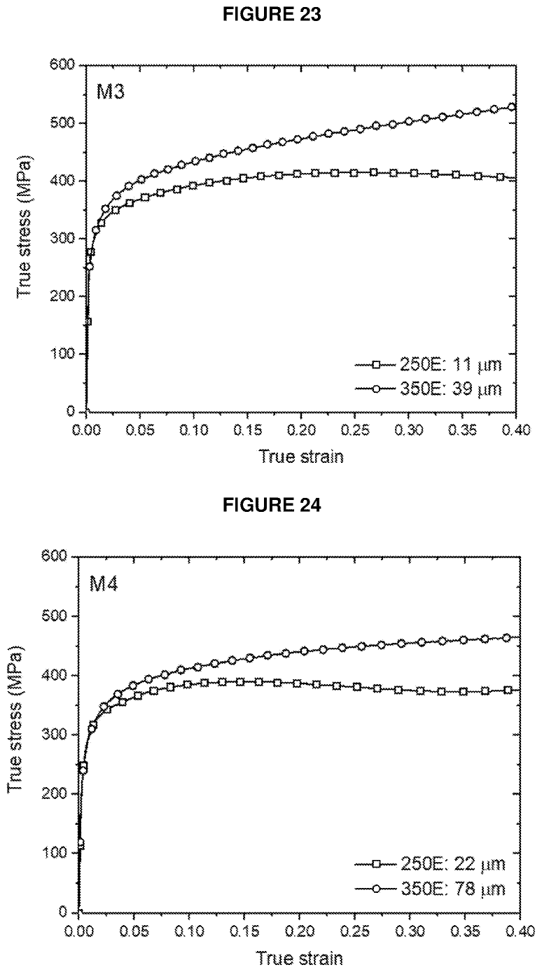

Samples of M3 and M4 alloys extruded at two different temperatures were subjected to uniaxial compression and the true stress-strain curves are shown in FIGS. 23 and 24. The compressive yield strength values of the extruded M3 and M4 alloy samples are listed in Table 12. When the extrusion temperature was increased from 250 °C to 350 °C the alloy compressive yield strength increased for both M3 and M4 alloys.

TABLE 12

Designation

| | |
|---|---|
| Average | Compressive |
| grain size | YS |
| (μm) | (MPa) |

M3

| | | |
|---|---|---|
| 250E | 11 | 277 |
| 350E | 39 | 294 |

M4

| | | |
|---|---|---|
| 250E | 22 | 250 |
| 350E | 78 | 266 |

### Example 10 (Comparative): Effect of Extrusion Temperature on Annealing Strengthening Response of M3 and M4 Alloy

The effect of extrusion temperature on annealing strengthening response was investigated in the M3 and M4 alloys. The same homogenization conditions prior to extrusion as in Examples 1 and 2 were used. The homogenized M3 alloy was subjected to extrusion at 115 °C, and the homogenized M4 alloy was extruded at 165 °C. The extrusion ratio was controlled of 36:1, and pushing rod speed was controlled of 0.1 mm·s⁻¹. The extrusion and post-annealing conditions are listed in Table 13.

TABLE 13

| Designation | | | |
|---|---|---|---|
| Extrusion | | Post-extrusion annealing | |
| Temperature | Extrusion | Temperature | Time |
| (°C) | ratio | (°C) | (h) |

M3

| | | | | |
|---|---|---|---|---|
| 175E | 175 | 36:1 | — | — |
| 175E + 250 °C/2 h | 175 | 36:1 | 250 | 2 |
| 175E + 300 °C/2 h | 175 | 36:1 | 300 | 2 |
| 175E + 350 °C/2 h | 175 | 36:1 | 350 | 2 |

M4

| | | | | |
|---|---|---|---|---|
| 165E | 165 | 36:1 | — | — |
| 165E + 250 °C/2 h | 165 | 36:1 | 250 | 2 |
| 165E + 300 °C/2 h | 165 | 36:1 | 300 | 2 |
| 165E + 350 °C/2 h | 165 | 36:1 | 350 | 2 |

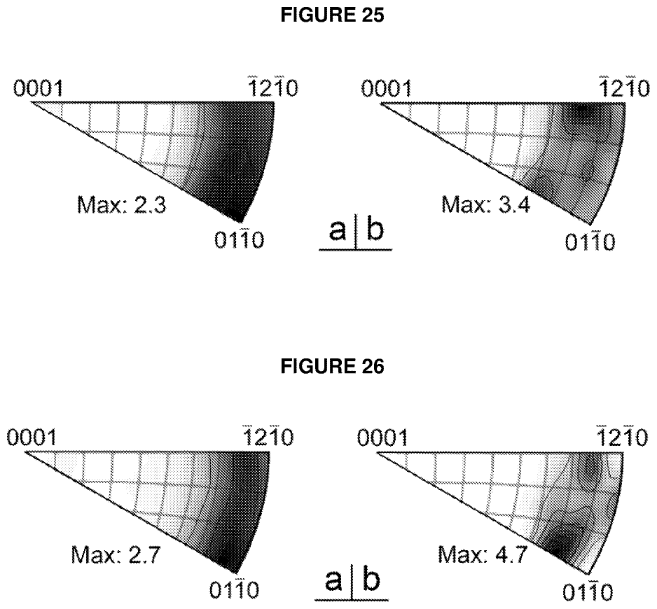

After the hot extrusion at 175 °C for the M3 alloy and 165 °C for the M4 alloy, post-extrusion annealing treatments were conducted at 250 °C, 300 °C, and 350 °C for 2 hours. Both the 175 °C extruded M3 and 165 °C extruded M4 alloys subjected to post-extrusion annealing treatment at 300 °C exhibited a non-basal texture as shown in FIG. 25 (wherein (a) is the extruded M3 alloy and (b) is the subsequently annealed M3 alloy) and FIG. 26 (wherein (a) is the extruded M4 alloy and (b) is the subsequently annealed M4 alloy).

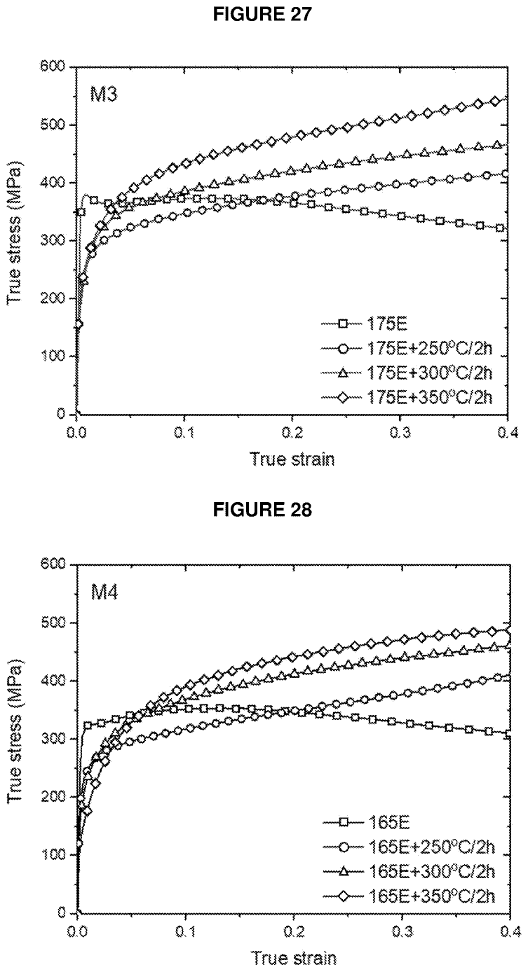

The as-extruded samples as well as samples annealed under three different conditions were subjected to uniaxial compression and the true stress-strain curves are shown in FIGS. 27 and 28 respectively for the M3 and M4 alloys. The compressive yield strength values of the extruded and annealed M3 and M4 alloy samples are listed in Table 14. It was found that when the extrusion temperature is decreased to 175 °C for the M3 alloy and 165 °C for the M4 alloy it eliminated the annealing strengthening behaviour. For the M3 and M4 alloys extruded at 175 °C and 165 °C, respectively, the as-extruded samples and the samples subjected to post-extrusion annealing at 300 °C for 2 hours, were evaluated under uniaxial tensile testing at room temperature and a strain rate of 10⁻³ s⁻¹, and their tensile yield strength values are listed in Table 14. There was no annealing strengthening response in the tensile deformation.

TABLE 14

Designation

| | | |
|---|---|---|
| Average | Compressive | Tensile |
| grain size | YS | YS |
| (μm) | (MPa) | (MPa) |

M3

| | | | |
|---|---|---|---|
| 175E | 5 | 373 | 352 |
| 175E + 250 °C/2 h | 17 | 215 | — |
| 175E + 300 °C/2 h | 49 | 190 | 149 |
| 175E + 350 °C/2 h | 77 | 218 | — |

M4

| | | | |
|---|---|---|---|
| 165E | 3 | 311 | 301 |
| 165E + 250 °C/2 h | 34 | 212 | — |
| 165E + 300 °C/2 h | 71 | 185 | 146 |
| 165E + 350 °C/2 h | 556 | 131 | — |

### Example 11 (Comparative): Creep Test of a Mg-0.4Zn-0.1Ca Alloy

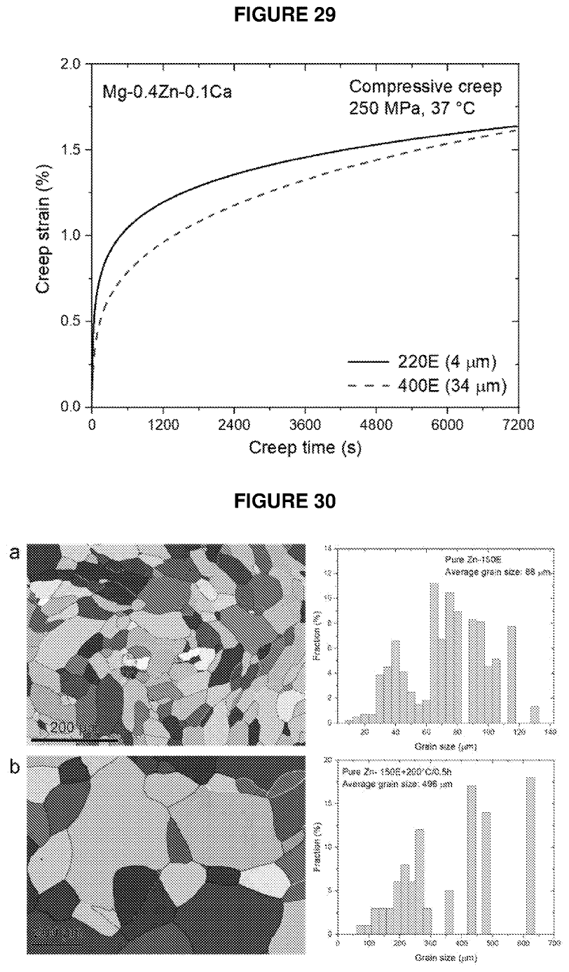

The creep curves of a dilute magnesium alloy of formula Mg-0.4Zn-0.1Ca (comprising nominally 0.4 wt. % Zn and 0.1 wt. % Ca) extruded at either 220 °C or 400 °C and examined under a loading stress of 250 MPa at 37 °C (human body temperature) are shown in FIG. 29. The creep resistance of the presently disclosed dilute zinc alloys is exceptional compared to that of the Mg-0.4Zn-0.1Ca alloy under the same creep condition.

### Example 12 (Comparative): Pure Zinc

Pure zinc was extruded into a cylindrical bar of 6 mm diameter at a temperature above 150 °C with a pushing rod speed of 0.1 mm·s⁻¹. The extrusion ratio was controlled to be about 36:1. After hot extrusion, annealing treatment was conducted at a temperature of 200 °C for 0.5 hours.

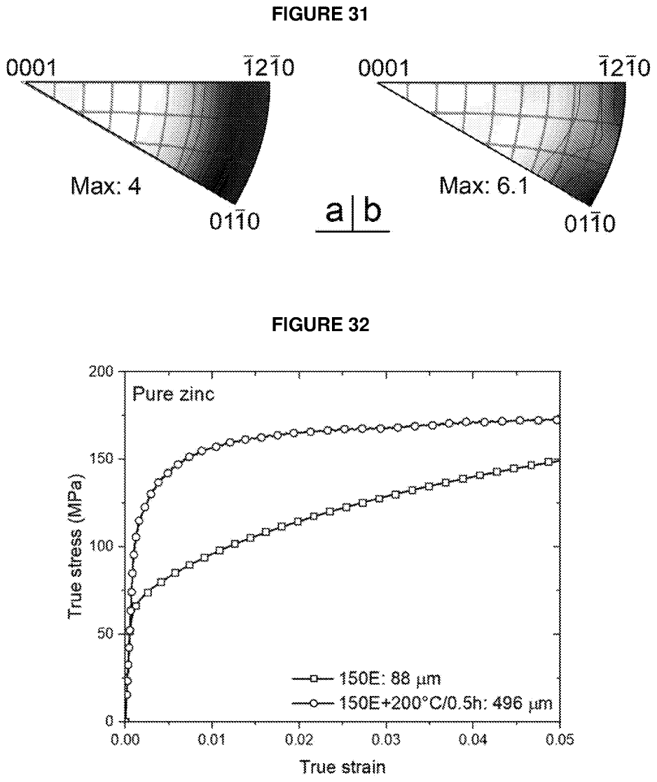

FIG. 30 shows the microstructures (left hand images) and grain size distributions (right hand images) of pure zinc, respectively (a) 150E and (b) 150E+200 °C/0.5 h. FIG. 31 shows the textures of the same two samples.

As-extruded and annealed samples were subjected to uniaxial compressive deformation along the extrusion direction at room temperature and a strain rate of 10⁻³ s⁻¹. The true stress-strain curves are shown in FIG. 32. The as-extruded alloy (150E) had a compressive yield strength of only 74 MPa, and the annealed sample (150E+200 °C/0.5 h) a compressive yield strength of only 132 MPa. The extruded sample of pure zinc was not strong enough to make or machine samples for tensile testing.

1. A process for preparing a dilute zinc alloy comprising the steps of:
(a) extruding homogenised dilute zinc alloy at a temperature above 175 °C; and
(b) optionally annealing the extruded dilute zinc alloy at a temperature from about 250 °C to about 400 °C;
wherein the dilute zinc alloy comprises at least 99.0 wt. % zinc; and one or both of iron and copper in total amount from about 0.01 wt. % to about 0.99 wt. %;
wherein the dilute zinc alloy comprises from about 0.01 wt. % to about 0.7 wt. % of one or more of magnesium, calcium, manganese, and lithium; and
wherein the sum of iron, copper, and one or more of magnesium, calcium, manganese, and lithium does not exceed about 1.0 wt. %.

2. The process according to claim 1, wherein the dilute zinc alloy comprises at least 99.0 wt. % zinc; and one or both of iron and copper in total amount from about 0.03 wt. % to about 0.78 wt. %;
wherein the dilute zinc alloy comprises from about 0.02 wt. % to about 0.7 wt. % of one or more of magnesium, calcium, manganese, and lithium; and
wherein the sum of iron, copper, and one or more of magnesium, calcium, manganese, and lithium does not exceed 0.8 wt. %.

3. The process according to claim 1, wherein the dilute zinc alloy comprises at least 99.0 wt. % zinc; and either,
from about 0.02 wt. % to about 0.97 wt. % iron and from about 0.01 wt. % to about 0.1 wt. % copper, or
from about 0.06 wt. % to about 0.5 wt. % copper and from about 0.01 wt. % to about 0.19 wt. % iron;
wherein the dilute zinc alloy comprises from about 0.01 wt. % to about 0.2 wt. % of one or more of magnesium, calcium, manganese, and lithium; and
wherein the sum of iron, copper, and one or more of magnesium, calcium, manganese, and lithium does not exceed 1.0 wt. %.

4. The process according to claim 1, wherein the dilute zinc alloy comprises at least 99.5 wt. % zinc; and either,
from about 0.02 wt. % to about 0.5 wt. % iron and from about 0.01 wt. % to about 0.06 wt. % copper, or
from about 0.06 wt. % to about 0.2 wt. % copper and from about 0.01 wt. % to about 0.05 wt. % iron;
wherein the dilute zinc alloy comprises from about 0.01 wt. % to about 0.2 wt. % of one or more of magnesium, calcium, manganese, and lithium; and
wherein the sum of iron, copper, and one or more of magnesium, calcium, manganese, and lithium does not exceed 0.5 wt. %.

5. The process according to claim 1 or claim 2, wherein the sum of iron, copper, and one or more of magnesium, calcium, manganese, and lithium does not exceed 0.7 wt. %, or 0.6 wt. %, or 0.5 wt. %, or 0.4 wt. %, or 0.3 wt. %.

6. The process according to any one of claims 1 to 5, wherein the dilute zinc alloy has a strong basal texture post-extrusion.

7. The process according to any one of claims 1 to 6, wherein the dilute zinc alloy has a strong basal-texture post annealing.

8. The process according to any one of claims 1 to 7, wherein extrusion is performed at a temperature above 200 °C.

9. The process according to any one of claims 1 to 7, wherein extrusion is performed at a temperature from about 200 °C to about 380 °C.

10. The process according to any one of claims 1 to 9, wherein extrusion speed is from about 0.01 mm·s⁻¹ to about 10 mm·s⁻¹, or from about 0.05 mm·s⁻¹ to about 0.5 mm·s⁻¹.

11. The process according to any one of claims 1 to 10, wherein extrusion ratio is from about 8:1 to about 80:1, or from about 19:1 to about 50:1.

12. The process according to any one of claims 1 to 11, wherein the annealing is performed at a temperature from about 250 °C to about 350 °C.

13. The process according to any one of claims 1 to 12, wherein the annealing is performed from about 0.5 hours to about 50 hours, or from about 1 hour to about 10 hours.

14. The process according to any one of claims 1 to 13, comprising a homogenisation step prior to extrusion wherein the dilute zinc alloy is maintained at a temperature from about 300 °C to about 400 °C for a period up to about 5 hours.

15. The process according to any one of claims 1 to 14, wherein an average grain size of the post-extruded dilute zinc alloy is greater than 5 micron, or greater than 10 micron.

16. The process according to any one of claims 1 to 15, wherein an average grain size of the post-extruded dilute zinc alloy is less than 25 micron.

17. The process according to any one of claims 1 to 16, wherein the annealing process increases an average grain size of the dilute zinc alloy.

18. The process according to any one of claims 1 to 17, wherein an average grain size of the post-annealed dilute zinc alloy is greater than 25 micron.

19. The process according to any one of claims 1 to 17, wherein an average grain size of the post-annealed dilute zinc alloy is from about 25 micron to about 1000 micron.

20. The process according to any one of claims 1 to 19, wherein the annealing process increases the dilute zinc alloy's ambient temperature compressive yield strength.

21. The process according to any one of claims 1 to 20, wherein the annealing process decreases the dilute zinc alloy's minimum creep rate.

22. The process according to any one of claims 1, 2, or 5 to 21, wherein the dilute zinc alloy comprises:
(a) at least 99.0 wt. % zinc;
(b) about 0.01 wt. % iron;
(c) about 0.04 wt. % copper; and
(d) about 0.05 wt. % magnesium;
or
(a) at least 99.0 wt. % zinc;
(b) about 0.06 wt. % copper; and
(c) about 0.04 wt. % magnesium;
or
(a) at least 99.0 wt. % zinc;
(b) about 0.06 wt. % iron;
(c) about 0.14 wt. % copper;
(d) about 0.01 wt. % calcium;
(e) about 0.09 wt. % manganese; and
(f) about 0.16 wt. % magnesium;
or
(a) at least 99.0 wt. % zinc;
(b) about 0.01 wt. % iron;
(c) about 0.37 wt. % copper;
(d) about 0.05 wt. % manganese; and
(e) about 0.18 wt. % magnesium;
or
(a) at least 99.0 wt. % zinc;
(b) about 0.02 wt. % iron;
(c) about 0.1 wt. % copper;
(d) about 0.02 wt. % calcium;
(e) about 0.16 wt. % manganese; and
(f) about 0.15 wt. % magnesium.

23. A dilute zinc alloy formed by the process according to any one of claims 1 to 22.

24. A dilute zinc alloy comprising:
at least 99.0 wt. % zinc; and one or both of iron and copper in total amount from about 0.01 wt. % to about 0.99 wt. %;
wherein the dilute zinc alloy comprises from about 0.01 wt. % to about 0.7 wt. % of one or more of magnesium, calcium, manganese, and lithium; and
wherein the sum of iron, copper, and one or more of magnesium, calcium, manganese, and lithium does not exceed about 1.0 wt. %.

25. The dilute zinc alloy according to claim 24, wherein the dilute zinc alloy comprises:
at least 99.0 wt. % zinc; and one or both of iron and copper in total amount from about 0.03 wt. % to about 0.78 wt. %;
wherein the dilute zinc alloy comprises from about 0.02 wt. % to about 0.7 wt. % of one or more of magnesium, calcium, manganese, and lithium; and
wherein the sum of iron, copper, and one or more of magnesium, calcium, manganese, and lithium does not exceed 0.8 wt. %.

26. The dilute zinc alloy according to claim 24 comprising:
(a) at least 99.0 wt. % zinc;
(b) either, from about 0.02 wt. % to about 0.97 wt. % iron and from about 0.01 wt. % to about 0.1 wt. % copper, or from about 0.06 wt. % to about 0.5 wt. % copper and from about 0.01 wt. % to about 0.19 wt. % iron;
(c) from about 0.01 wt. % to about 0.12 wt. % of one or more of magnesium, calcium, manganese, and lithium;
wherein the sum of iron, copper, and one or more of magnesium, calcium, manganese, and lithium does not exceed 1.0 wt. %.

27. The dilute zinc alloy according to claim 24, wherein the dilute zinc alloy comprises:
(a) at least 99.5 wt. % zinc;
(b) either, from about 0.02 wt. % to about 0.5 wt. % iron and from about 0.01 wt. % to about 0.06 wt. % copper, or from about 0.06 wt. % to about 0.2 wt. % copper and from about 0.01 wt. % to about 0.05 wt. % iron;
(c) from about 0.01 wt. % to about 0.12 wt. % of one or more of magnesium, calcium, manganese, and lithium;
wherein the sum of iron, copper, and one or more of magnesium, calcium, manganese, and lithium does not exceed 0.5 wt. %.

28. The dilute zinc alloy according to claims 24 or 25, wherein the sum of iron, copper, and one or more of magnesium, calcium, manganese, and lithium does not exceed 0.7 wt. %, or 0.6 wt. %, or 0.5 wt. %, or 0.4 wt. %, or 0.3 wt. %.

29. The dilute zinc alloy according to any one of claims 24 to 28, wherein the dilute zinc alloy has a strong basal texture.

30. The dilute zinc alloy according to any one of claims 24 to 29, wherein an average grain size of the dilute zinc alloy is greater than 5 micron, or greater than 10 micron, or greater than 25 micron.

31. The dilute zinc alloy according to any one of claims 24 to 29, wherein an average grain size of the dilute zinc alloy is from about 5 micron to about 1000 micron.

32. The dilute zinc alloy according to any one of claims 24 to 31, wherein a compressive strength of the dilute zinc alloy is greater than 220 MPa when measured at ambient temperature and a strain rate of 10⁻³ s⁻¹, or greater than 230 MPa, or greater than 240 MP a, or greater than 250 MP a, or greater than 260 MPa, or greater than 270 MP a, or greater than 280 MP a, or greater than 290 MP a, or greater than 300 MP a.

33. The dilute zinc alloy according to any one of claims 24 to 32, wherein a minimum compressive creep rate of the dilute zinc alloy is less than 3×10⁻⁶ s⁻¹ under a loading stress of 200 MPa at 37 °C, or less than 2×10⁻⁶ s⁻¹, or less than 1×10⁻⁶ s⁻¹, or less than 5×10⁻⁷ s⁻¹, or less than 3×10⁻⁷ s⁻¹, or less than 2×10⁻⁷ s⁻¹, or less than 1×10⁻⁷ s⁻¹.

34. The dilute zinc alloy according to any one of claims 24, 25, or 28 to 33, wherein the dilute zinc alloy comprises:
(a) at least 99.0 wt. % zinc;
(b) about 0.01 wt. % iron;
(c) about 0.04 wt. % copper; and
(d) about 0.05 wt. % magnesium;
or
(a) at least 99.0 wt. % zinc;
(b) about 0.06 wt. % copper; and
(c) about 0.04 wt. % magnesium;
or
(a) at least 99.0 wt. % zinc;
(b) about 0.06 wt. % iron;
(c) about 0.14 wt. % copper;
(d) about 0.01 wt. % calcium;
(e) about 0.09 wt. % manganese; and
(f) about 0.16 wt. % magnesium;
or
(a) at least 99.0 wt. % zinc;
(b) about 0.01 wt. % iron;
(c) about 0.37 wt. % copper;
(d) about 0.05 wt. % manganese; and
(e) about 0.18 wt. % magnesium;
or
(a) at least 99.0 wt. % zinc;
(b) about 0.02 wt. % iron;
(c) about 0.1 wt. % copper;
(d) about 0.02 wt. % calcium;
(e) about 0.16 wt. % manganese; and
(f) about 0.15 wt. % magnesium.

35. An article of manufacture comprising the dilute zinc alloy according to any one of claims 24 to 34.

36. The article of manufacture according to claim 35, wherein the article is a medical implant.

37. The article of manufacture according to claim 36, wherein the medical implant is a stent, a plate, a screw, a nail, a pin, or a scaffold.
